(19) United States
(12) Patent Application Publication
(10) Pub. No.: US 2011/0112373 A1
(43) Pub. Date: May 12, 2011

(54) SOFT TISSUE ACCESS APPARATUS AND METHODS FOR SPINAL SURGERY

(75) Inventors: Stephen D. Ainsworth, Wilmington, NC (US); Eugene E. Avidano, Wilmington, NC (US); Frederic C. Feiler, JR., Wilmington, NC (US)

(73) Assignee: TRANS1 INC., Wilmington, NC (US)

(21) Appl. No.: 12/916,463

(22) Filed: Oct. 29, 2010

Related U.S. Application Data
(60) Provisional application No. 61/259,977, filed on Nov. 10, 2009.

(51) Int. Cl. A61B 1/32

(52) U.S. Cl. 600/207

(57) ABSTRACT

Disclosed are surgical tools, tool sets and methods for percutaneously accessing and preparing treatment sites within the spine for subsequent treatment procedures. The treatment site may be avertebral motion segments in the lumbar and sacral regions of the spine. The tool set may a retraction device for retracting bowel from a sacrum. The device can induce a collapsible enclosure that defines a chamber. The collapsible enclosure can have a collapsed configuration and an expanded configuration. In some arrangements, the chamber is filled with an inflation media. The expanded configuration defines a working channel positioned between the sacrum and the bowel and extending over or through at least a portion of the device.

### CROSS-REFERENCE TO RELATED APPLICATIONS

The present application claims priority under 35 U.S.C. §119(e) to U.S. Provisional Application No. 61/259,977 filed on Nov. 10, 2009, the entire disclosure of which is incorporated by reference herein in its entirety.

### TECHNICAL FIELD

The present application is generally related to surgical methods and devices, and more particularly to methods and devices for providing soft tissue access to a target site in a patient.

### BACKGROUND

U.S. Pat. No. 7,530,993 describes surgical tools, tool sets and methods for percutaneously accessing and preparing treatment sites on the sacrum of the spine. As described in this patent, after the spine is accessed, various devices and tools can be inserted into the spine trans-sacrally in order to treat various spinal disorders and injuries. Examples of such procedures and associated devices and tools are disclosed in U.S. Provisional Patent Application No. 60/182,748, filed Feb. 16, 2000, U.S. patent application Ser. No. 09/640,222, filed on Aug. 16, 2000 now U.S. Pat. No. 6,575,979, U.S. patent application Ser. No. 09/782,583 filed on Feb. 13, 2001, now U.S. Pat. No. 6,558,390, U.S. patent application Ser. No. 09/848,556 filed on May 3, 2001, now U.S. Pat. No. 7,014,633, U.S. patent application Ser. No. 10/125,771 filed on Apr. 18, 2002, now U.S. Pat. No. 6,899,716, U.S. patent application Ser. No. 10/309,416 filed on Dec. 3, 2002, now U.S. Pat. No. 6,921,403, U.S. patent application Ser. No. 10/972,065, filed on Oct. 22, 2004, U.S. patent application Ser. No. 11/189,943 filed Jul. 26, 2005, and U.S. patent application Ser. No. 11/501,351, filed on Aug. 9, 2006, the contents of each of which are incorporated in their entirety into this disclosure by reference herein.

The trans-sacral approach to lumbar surgery described in the above-referenced patents and patent applications represents a pioneering and innovative approach to spinal surgery. In addition, the surgical tools and methods described in these references provide for a minimally invasive and reproducible approach for providing access to primarily, but not limited to, the L4-L5 and L5-S1 vertebral bodies. The devices and methods described above are commercially available and are made by TranS1, Inc. and sold under the trademarks AxiaLIF®, AxiaLIF 2L+™, AxiaLIF 2L®, AxiaLIF 360° ®, and TRANS1 VECTRE™. However, as with any surgical tools and methods, there remains a need to continuingly improve such tools and methods.

### SUMMARY

In one embodiment, a retraction device for retracting bowel from a sacrum includes a collapsible enclosure that defines a chamber. The collapsible enclosure has a collapsed configuration and an expanded filled configuration when the chamber is filled with a fluid. The expanded filled configuration defines a working channel positioned between the sacrum and the bowel and that extends over or through at least a portion of the device.

In one embodiment, of providing access to a target site on the sacrum of the patient, an inserter carrying an inflatable device is inserted into a presacral space between a bowel and a sacrum with a valve and at least a portion of an inflation tube coupled to the inflatable device. The valve is attached attaching to a source of inflation media. The inflatable device is filled with the inflation media to expand the collapsible enclosure and retract the bowel from the sacrum.

In one embodiment, a method of providing access to a target site on a sacrum of a patient; includes inserting at least part of a retraction device comprising an inflatable structure that defines a channel into a presacral space between a bowel of the patient and the sacrum and inflating the inflatable structure with an inflation media, such that the inflatable enclosure expands to retract the bowel from the sacrum and enlarge the channel defined through the retraction device to define a working space.

In one embodiment, a method of providing access to a target site on a sacrum of a patient; includes inserting at least part of a retraction device comprising an inflatable structure that defines a channel into a presacral space between a bowel of the patient and the sacrum and inflating the inflatable structure with an inflation media, such that the inflatable enclosure expands to retract the bowel from the sacrum and enlarge the channel defined through the retraction device to define a working space.

In one embodiment, a kit for providing retracting bowel from a sacrum includes an inflatable retraction device, an inflation device, an insertion tool, a tubular retractor and a fixation wire.

In an embodiment, an inflatable device for retracting bowel from a sacrum includes a collapsible enclosure that defines a chamber, the collapsible enclosure having an expanded inflated configuration and a deflated configuration. In some embodiments, a channel can be formed through the device in the expanded inflated configuration.

In an embodiment, the inflatable device also includes an elongated inserter that has a distal end and a proximal end, the proximal end of the inserter including a handle and the distal end of the inserter coupled to the collapsible enclosure. In an embodiment, the inflatable device also includes an interface on the collapsible enclosure, wherein the inserter can be coupled to the interface to facilitate insertion of the inflatable device into a presacral space. In an embodiment, the interface is a coupled to an exterior surface of the enclosure. In an embodiment, the collapsible enclosure has a width that is measured perpendicular to the longitudinal axis of the inserter, the width being greater than about 5 centimeters and less than about 8 centimeters. In an embodiment, the collapsible enclosure has a length that is parallel to the longitudinal axis of the inserter, the length being greater than about 7 centimeters and less than about 26 centimeters. In an embodiment, the collapsible enclosure in the expanded inflated configuration has a generally flat rectangular shape that has a depth, a width and a length. In an embodiment, the depth is less than the width, which is less than the length. In an embodiment, the length of collapsible enclosure is oriented along a longitudinal axis of the elongated inserter. In an embodiment, the collapsible enclosure in the expanded inflated configuration comprises a distal portion with a generally flat rectangular shape and a proximal portion with a generally flat trapezoidal shape. In an embodiment, the collapsible enclosure has a depth, a first width, a second width and a length. In an embodiment, the depth is less than the first width which is less than the length. In an embodiment, the first width is disposed along the distal portion and is generally constant. In an embodiment, the second width is disposed along the proximal portion and is tapered inwardly in the distal to proximal direction. In an embodiment, the length of collapsible enclosure is orientated along a longitudinal axis of the elongated inserter. In an embodiment, the collapsible enclosure has a first side and a second side that extend in a longitudinal direction that is generally parallel to a longitudinal axis of the inserter and wherein the inserter is positioned between the first and second sides. In an embodiment, the first and second sides are folded towards the inserter when the collapsible enclosure is in the deflated configuration and expand away from the inserter in the inflated configuration. In an embodiment, the inflatable device also includes a surgical tool for insertion between the collapsible enclosure and the sacrum and/or soft tissue. In an embodiment, the inflatable device also includes an elongate tube in fluid communication with the chamber. In an embodiment, the inflatable device also includes a valve connected to the elongate tube. In an embodiment, the surface of the enclosure is baffled or corrugated. In an embodiment, the inflatable device also includes one or more chambers within the enclosure. In an embodiment, the one or more chambers can be independently filled and emptied through one or more inflation lines.

In an embodiment, a method of providing access to a target site on the sacrum of the patient includes the steps of inserting a retraction device comprising an inflatable structure into a presacral space between a bowel of the patient and the sacrum, inflating the inflatable structure with an inflation media such that the inflatable enclosure expands to retract the bowel from the sacrum, and inserting a surgical tool into the space created between the inflatable structure and the sacrum.

In an embodiment, the method also includes at least partially emptying the inflatable structure of the inflation media, repositioning the inflatable structure within the presacral space, and refilling the chamber with inflation media. In an embodiment, the step of inserting the inflatable structure into a presacral space includes positioning the inflatable structure adjacent a sacral promontory. In other embodiments, the inflatable structure can be repositioned without deflating the inflatable structure.

In an embodiment, a method of providing access to a target site on the sacrum of the patient includes the steps of inserting an inserter carrying an inflatable device into a presacral space between the bowel and the sacrum such that a valve and at least a portion of an inflation tube is coupled to the inflatable device with the inflation tube, attaching the valve to a source of inflation media, and filling the chamber with the inflation media to expand the collapsible enclosure and retract the bowel from the sacrum.

In an embodiment, the method also includes the steps of at least partially emptying the inflatable device of the inflation media, repositioning the inflatable device within the presacral space by manipulating the inserter, refilling the chamber with inflation media, and removing the inserter from the inflation device. In an embodiment, the step of inserting the inflatable device into a presacral space includes positioning the inflatable device adjacent a sacral promontory. In an embodiment, the method also includes inserting a surgical tool into the space created between the inflatable device and the sacrum. In certain embodiments, the inflatable device can be repositioned without deflating the device and/or with the device partially inflated.

In some embodiments, a method of providing access to a target site on a sacrum of a patient includes the steps of inserting at least part of a retraction device comprising an inflatable structure into a presacral space between a bowel of the patient and the sacrum, and inflating the inflatable structure with an inflation media, such that the inflatable enclosure expands to retract the bowel from the sacrum and create a working channel through or over the retraction device. Surgical tools can be inserted through the working channel to the sacrum.

Some embodiments include an inflatable structure that defines, at least in part, a 360 degrees protected portal for the passage of instrumentation from an incision site to a target site on a sacrum. In some arrangements, the inflatable structure can also dissect tissue as it is inflated.

In certain embodiments, the tools and devices described above can be arranged into kits or assemblies in which the tools and devices are packaged and/or assembledtogether and/or used in combination.

These and other advantages and features of the surgical tools, kits, assemblies, methods and techniques disclosed in the embodiments of the present invention will be more readily understood from the following detailed description of the preferred embodiments thereof, when considered in conjunction with the accompanying figures and claims.

### BRIEF DESCRIPTION OF THE DRAWINGS

FIG. 1 illustrates a side view of one embodiment of a tissue retractor implanted in a presacral space.

FIG. 1A is a lateral view of the lumbar and sacral portion of the spinal column depicting a visualized anterior axial instrumentation/implant line (AAIIL) extending cephalad and axially from an anterior site target point according to one embodiment

FIG. 1B is an illustration of an anterior target point on the sacrum according to one embodiment.

FIGS. 1C and 1D are cross-sectional caudal views of a lumbar vertebra depicting one and two trans sacral axial implants, respectively, within corresponding TASII bores formed in parallel with the visualized AAIIL of FIG. 1A.

FIG. 2 is a schematic side sectional view of the anatomy of a portion of the body including a presacral space.

FIG. 3 is a schematic front sectional view of a rectum and anus.

FIG. 4 is a schematic partial sectional view of portions of the anatomy near the anus.

FIGS. 5A-5C are schematic side sectional views of steps in accessing the anatomy of a portion of the body including a presacral space according to one embodiment.

FIGS. 6A-D illustrate various schematic views of a guide pin introducer according to an embodiment.

FIGS. 7A-D illustrate various schematic views of a stylet according to an embodiment.

FIGS. 8A and 8B are lateral, partial cross-sectional views of the lumbar and sacral portion of the spine depicting delivery of the distal end of guide pin introducer-stylet-assembly to the anterior surface of the S1 vertebral body according to one embodiment.

FIG. 9 illustrates a top perspective view of one embodiment of a tissue retractor.

FIG. 10 illustrates a top perspective view of an embodiment of a tissue retractor.

FIG. 11A illustrates a schematic side sectional view of an embodiment of a tissue retractor implanted in a presacral space.

FIG. 11B illustrates a schematic side sectional view of an embodiment of an inflated tissue retractor with an introducer assembly.

FIGS. 12A-B illustrate top plan views of a tissue retractor according to another embodiment.

FIG. 13 illustrates a schematic side sectional view of an inflated tissue retractor according to an embodiment.

FIG. 14A illustrates a top perspective view of another embodiment of a tissue retractor with a comfortable tip.

FIG. 14B illustrates a top perspective view of the tissue retractor in FIG. 14A in combination with a tubular retractor.

FIG. 14C illustrates a side view of the tissue retractor in FIG. 14A.

FIG. 15A illustrates a top perspective view of a tissue retractor according to another embodiment.

FIG. 15B illustrates a top perspective view of the tissue retractor in FIG. 15A in combination with a tubular retractor having a conformable tip.

FIGS. 16A-C illustrate various schematic views of a tissue retractor according to another embodiment.

FIG. 17A illustrates a rear top perspective view of another embodiment of a tissue retractor.

FIG. 17B illustrates a top view of the tissue retractor in FIG. 17A.

FIG. 17C illustrates a front top perspective view of the tissue retractor in FIG. 17A.

FIG. 17D illustrates a top plan view of the tissue retractor in FIG. 17A in a collapsed configuration.

FIG. 18A illustrates a perspective side schematic views of a tissue retractor according to another embodiment.

FIG. 18B illustrates a side view of the tissue retractor of FIG. 18B.

FIG. 19A illustrates a top perspective view of an insertion tool according to an embodiment.

FIG. 19B illustrates an exploded top perspective view of the insertion tool of FIG. 19A.

FIG. 20A illustrates a top perspective view of an insertion tool according to an embodiment.

FIG. 20B illustrates an exploded top perspective view of the insertion tool of FIG. 20A.

FIG. 20C illustrates an exploded bottom perspective view of the insertion tool of FIG. 20A.

FIG. 20D illustrates closer bottom perspective view of a proximal portion of the insertion tool of FIG. 20A.

FIG. 21 illustrates a schematic side sectional view of an embodiment of an inflated tissue retractor implanted in a presacral space, in combination with a tubular retractor.

FIG. 22 illustrates a schematic side sectional view of another embodiment of an inflated tissue retractor implanted in a presacral space.

FIG. 23 illustrates a schematic side sectional view of an inflated tissue retractor according to an embodiment implanted in a presacral space, in combination with a guide wire.

FIG. 24 illustrates a schematic side sectional view of an inflated tissue retractor according to another embodiment implanted in a presacral space, in combination with an introducer assembly.

FIGS. 25A-B illustrate a schematic side sectional view of a tissue retractor being inserted in a presacral space using an insertion tool according to another embodiment.

FIG. 26 illustrates several tools that can be inserted through or over a tissue retractor.

### DETAILED DESCRIPTION

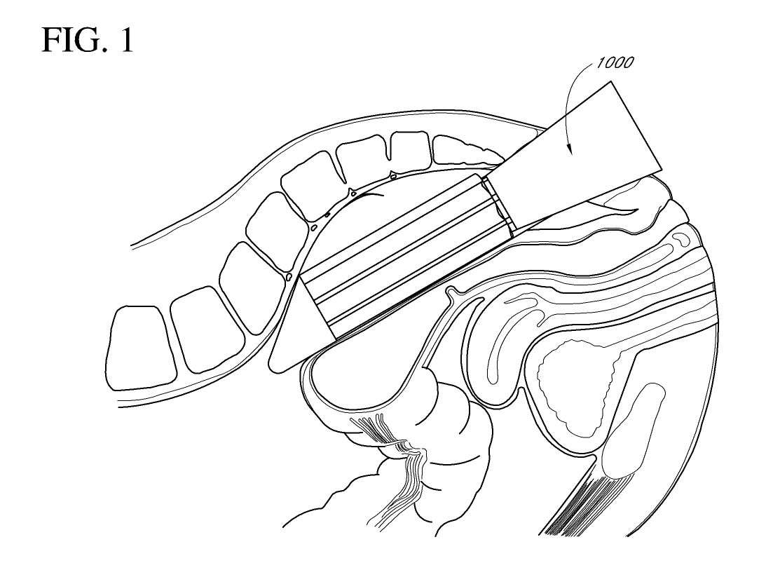

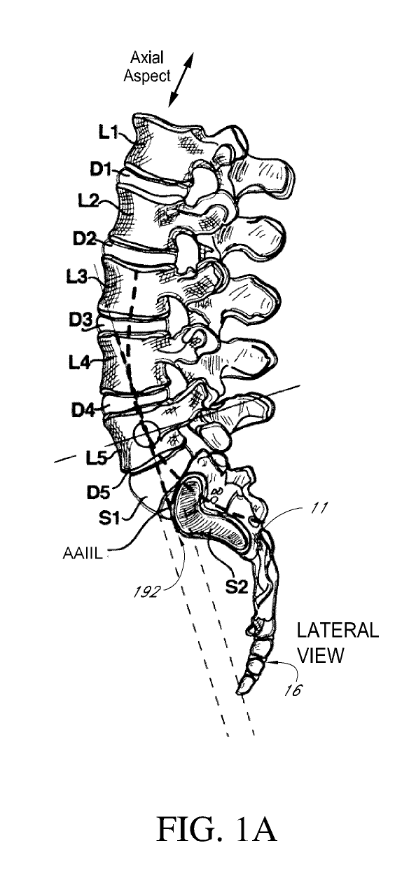

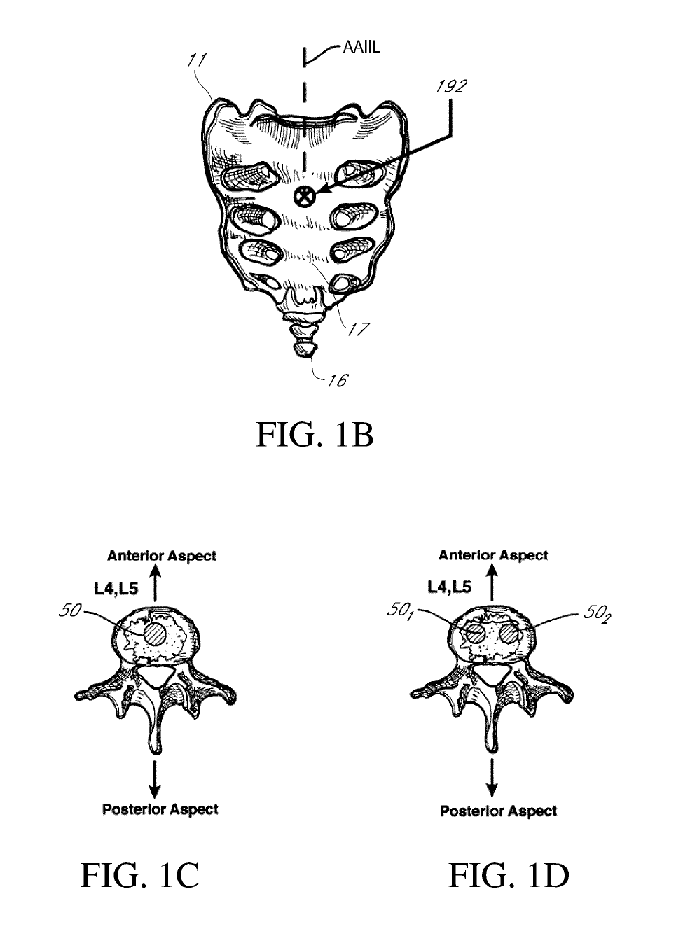

FIG. 1 illustrates a tissue retraction device 1000 according to one embodiment. As will be explained in detail below, the tissue retraction device 1000 can be inflated or otherwise expanded to define a working space or channel that is generally positioned between the bowel and the sacrum. The working space or channel created by the tissue retraction device 1000 can form a portal that extends from a target site on a patient's sacrum towards or to an surgical access site such that tools and instruments can be inserted from the surgical access site, through the portal and to the target site. In these embodiments, the tissue retraction device 1000 can serve to protect the patient's soft tissue (e.g., the bowel) as the instruments are advanced towards the target site on the sacrum. In certain embodiments, the tissue retraction device 1000 can provide substantially 360 degrees (about the longitudinal axis of the device) of protection about the portal. In this manner, as tools are advanced towards the access site the tissue retraction device 1000 completely surrounds such tools preventing the tools from contacting or traumatizing the soft tissues of the patient. In other embodiments, the tissue retraction device can form an atraumatic barrier between the tools and the bowel as tools are passed over or partially through the device. In certain embodiments, the tissue retraction device 1000 can also retract the bowel from the sacrum and/or dissect tissue. An advantage of certain embodiments is that the tissue retractor can conform to the face of the sacrum.

Accordingly, in certain embodiments described herein, the tissue retraction device can form part of a surgical instrumentation system and technique for efficiently and atraumatically accessing, visualizing, and/or treating sites within a body, such as in the abdomen, sacrum, bowel, hip, and/or waist including sites in, on, around, near or proximate to a rectal, retrorectal, sacral and/or a presacral space. While in the illustrated embodiments the target site is the sacrum, in some embodiments the target site is soft tissue, such as organs, tumors, cysts or other bodily structures. In some embodiments a target site is hard tissue, such as bone. As noted above, in the illustrated embodiment, for example, the target site is the spine, such as, vertebral motion segments, for subsequent therapeutic spinal procedures. For example, in one embodiment, the spine (e.g., vertebral motion segments) is accessed for subsequent therapeutic procedures, such as, for example, spinal arthroplasty, partial or total disc replacement, annulus repair, vertebroplasty, arthrodesis (fusion), or the like. The therapeutic procedures can involve, among other things, facilitating the removal of tissue, preparing an intervertebral disc space for subsequent deployment of spinal fusion implants designed to relieve lower back pain, or motion preservation devices, e.g., dynamic stabilization devices, prosthetic nucleus devices or total disc replacements designed to relieve lower back pain and to restore physiological function of the spine, maintain and possibly improve disc health and prevent progression or transition of disease. In one approach, the step of accessing the treatment site can include using fluoroscopic imaging to visually align one or more components of the instrumentation system via a percutaneous, anterior trans-sacral axial approach. In another aspect, the treatment site includes a spinal disc and the subsequent therapeutic procedure includes nucleectomy. In yet another aspect, the therapeutic procedure includes immobilization devices to facilitate fusion; deployment of augmentation media; deployment of dynamic stabilization implants, or mobility devices to preserve or restore physiologic function. In one embodiment, the therapeutic procedures include the devices and associated methods that are commercially available or made by TranS1, Inc. and sold or made under the trademarks, e.g., AxiaLIF®, AxiaLIF 2L®, AxiaLIF 2L+™, AxiaLIF 360© and TRANS1 VECTRE™. Such therapeutic procedures include the devices and associated method described in, e.g., U.S. Pat. Nos. 7,776,042, 7,530,993 and 6,921,403 and U.S. Patent Publication No. 2007-0066977, the entire contents of these patents are hereby incorporated by reference herein. As will be described below, one or more of the tools and devices used in the treatments and therapeutic procedures described above can be inserted through the working channel or space created by the tissue retraction device.

In accordance with one aspect of the embodiments described herein, there are provided surgical tool sets and methods of using the tool sets. The tools of the tool sets can be used individually and/or in combination with each other.

It will be understood that the access methods described can include the step of utilizing a trans-sacral pathway using the working channel or by the tissue retraction device to perform certain techniques in the spine as mentioned above. More specifically the description herein will focus on using the tissue retraction device with techniques and procedures that utilize an anterior trans-sacral pathway.

For example, FIGS. 1A-1B schematically illustrate embodiments of the anterior trans-sacral axial spinal instrumentation/implant (TASII) approaches in relation to the lumbar region of the spinal column, and FIGS. 1C and 1D illustrate an embodiment of the location of a TASII implant or pair of implants within an anterior TASII axial bore 50 or pair of TASII axial bores 50₁, 50₂. Two TASII axial bores and spinal implants or rods are shown in FIG. 1D to illustrate that a plurality, that is two or more, of the same may be formed and/or employed in side by side relation parallel with the anterior axial instrumentation/implant line (AAIIL).

As context for the description herein, the lower regions of the spinal column comprising a coccyx 16, fused sacral vertebrae S1-S5 forming the sacrum 11, and the lumbar vertebrae L1-L5 described above are depicted in a lateral view in FIG. 1A. The series of adjacent vertebrae located within the human lumbar and sacral spine have an anterior aspect, a posterior aspect and an axial aspect, and the lumbar vertebrae are separated by intact or damaged spinal discs labeled D1-D5 in FIG. 1A. FIG. 1B depicts the anterior view of the sacrum 11 and coccyx 16.

In an embodiment, a method and apparatus for forming an anterior TASII axial bore initially involves accessing an anterior sacral position, e.g. an anterior target point at about the junction of S1 and S2 depicted in FIGS. 1A and 1B. One (or more) visualized, imaginary, axial instrumentation/implant line extends cephalad and axially in the axial aspect through the series of adjacent vertebral bodies to be fused or otherwise treated, L4 and L5 in this illustrated example. The visualized AAIIL through L4, D4, L5 and D5 extends relatively straight from the anterior target point along S1 depicted in FIGS. 1A and 1B, but may be curved as to follow the curvature of the spinal column in the cephalad direction.

It will be noted that the terms trans-sacral axial spinal instrumentation/implant (TASII), and anterior axial instrumentation/implant line (AAIIL), as used herein, are analogous to the terms trans-sacral axial spinal instrumentation/fusion (TASIF), and anterior axial instrumentation/fusion line (AAIFL). The analogous terms generally refer to the same percutaneous pathways, the primary difference being the types of treatments and implants delivered through the respective percutaneous pathways. In the description herein, the term “axial” when used with respect to the spine generally means along the longitudinal axis of the spinal column.

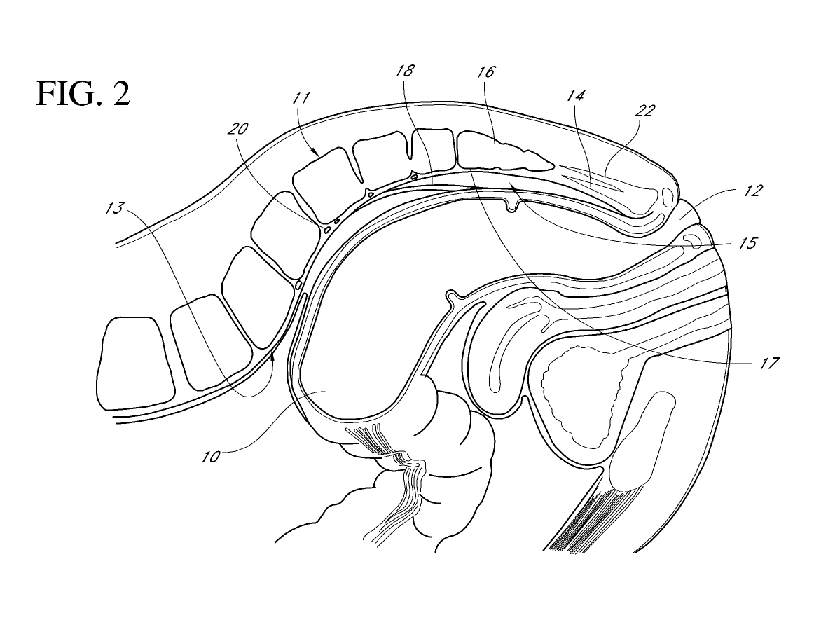

Referring to FIGS. 2-4, the rectum 10 terminates at the anus 12, and is 12 to 15 centimeters in length and its surgical boundaries consist of sacral promontory 13 proximally and pelvic floor muscles 14 distally. The rectum 10 is located anterior to the sacral concavity and ends approximately 2 to 3 centimeters anteriorly and inferiorly from the tip of the coccyx 16. The anococcygeal ligament 22 extends between the coccyx 16 and the anus 12. The sacrotuberous ligament 23 is also near the coccyx 16. The rectum 10 is covered by peritoneal lining only at the anterior and lateral portions of the upper rectum 10 and the rest is extra-peritoneal. The fascia propria or visceral layer of the pelvic fascia envelops the rectum 10 posteriorly and laterally in the extra-peritoneal portion of the rectum 10. This fascial layer is usually loosely attached to the presacral fascia.

The sacrum 11 and coccyx 16 can include a presacral fascia or the parietal layer of the pelvic fascia covers the concavity of the sacrum, coccyx 16, presacral veins 20, arteries, and nerves. Various muscles populate the space, including the internal sphincter muscle 26, the external sphincter muscle 28, the levator ani muscle 30, the gluteus maximus muscle 32, and the iliococcygeus muscle 34. Conventional operative dissection deep to the presacral fascia may result in troublesome bleeding.

In one embodiment, an access assembly provides access to the retrorectal or presacral space 15. The retrorectal or presacral space 15 is a potential space surrounded by fascia propria of the rectum 10 anteriorly, presacral fascia posteriorly, rectosacral ligament inferiorly, and retroperitoneum superiorly. Recto-sacral fascia or Waldeyer's fascia 18 is a thickened fascial reflection from the presacral fascia at S3-4 level to the fascia propria of the rectum 10 (FIG. 2). This fascial layer can be encountered during blunt dissection into the retrorectal or presacral space 15. In young patients, the Waldeyer's fascia 18 can be quite thickened and may require a sharp dissection in order to gain entry into the presacral space 15.

In accordance with some embodiments described herein, the patient can perform preoperative preparations prior to undertaking the disclosed surgical techniques. The preoperative preparations include standard preoperative procedures and preparations, as is known in the art.

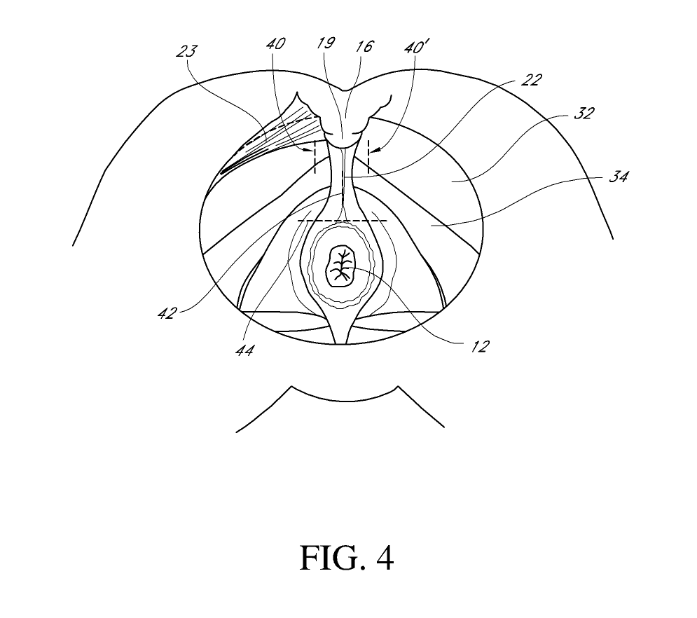

With reference to FIG. 4, in an embodiment the side of the approach (with incision 40 or 40′) is determined based on the surgeon's preference. The paracoccygeal notch 19 is palpated to determine the incision location. A 2-3 centimeter longitudinal paramedian/paracoccygeal incision 40, 40′ can be made. The incision 40, 40′ is typically 1-2 centimeter off midline; its superior aspect lies just below the paracoccygeal notch 19. In other embodiments, an incision can be a midline incision (vertical) 42. In an embodiment, an incision is an ano-coccygeal (transverse) incision 44. The skin and underling fascia are incised carefully, with cautious dissection into the presacral space 15. In one embodiment, an opening large enough to accommodate an index finger is created. In one embodiment a 2 centimeter paramedian incision is created just below the sacrotuberous ligament 23.

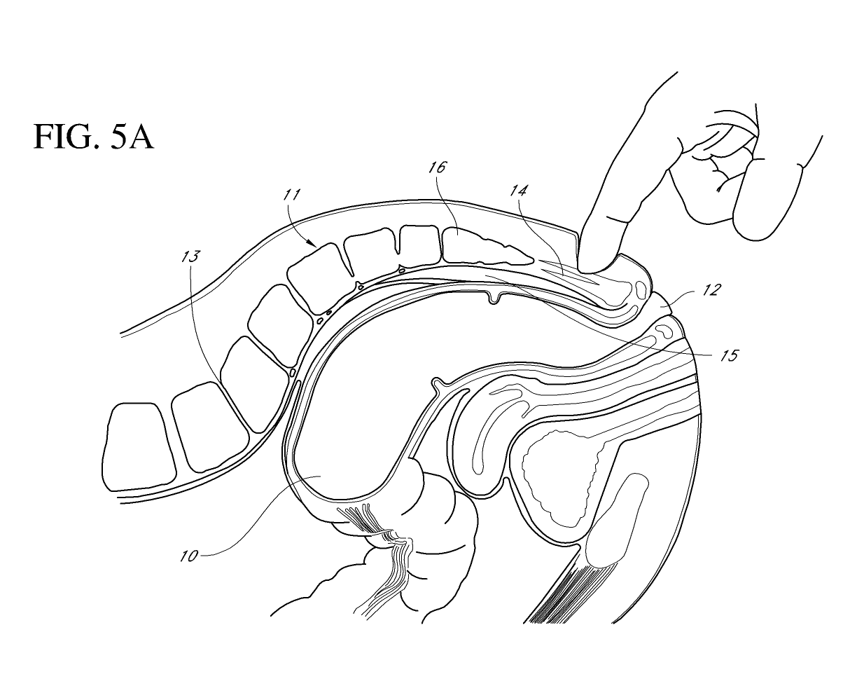

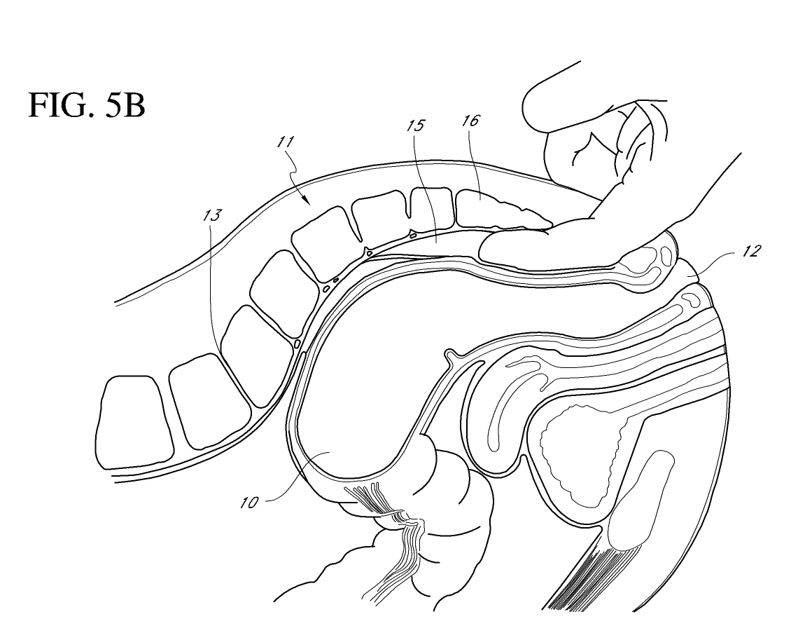

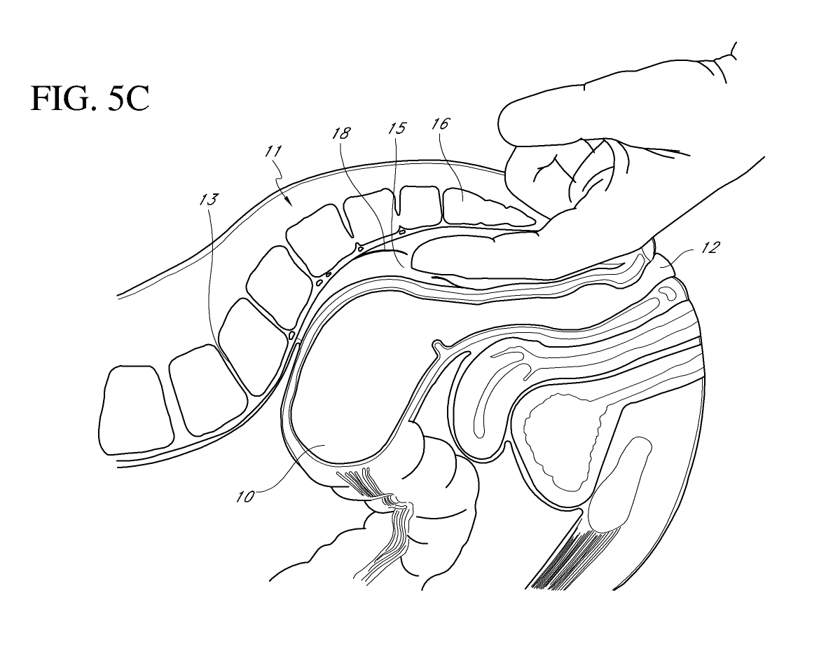

Referring to FIGS. 5A, 5B and 5C, in one embodiment, blunt finger dissection can be employed to dissect the plane between the rectum 10 and the sacrum 11. In one embodiment, a blunt finger dissection is called a “pop” through procedure. Both the rectum 10 and the sacrum 11 are enveloped in their own respective fascia: the rectum 10 is covered by the visceral fascia and the sacrum 11 is covered by the parietal fascia. The potential space in between these two fascial planes is the retrorectal or presacral space 15. The tissues in this plane are usually avascular and areolar in quality, very similar in composition to “cotton candy.” In one embodiment, blunt finger dissection can be performed through the subcutaneous tissues down to the pelvic floor complex just anterior to the coccyx 16. The finger sweeping dissection is continued in cephalad direction towards the sacral promontory 13. The surgeon should feel smooth surfaces on both the rectum 10 and the sacrum 11. A ragged, bumpy-like tissue surface should raise the suspicion of being in the wrong plane. This can present either as dissection that is too deep into the rectal fat or mesorectum or too deep into and below the sacral fascia. The dangers of being in the wrong dissection plane can be noted, with increased risk of rectal perforation or increased risk of presacral bleeding from the sacral venous plexus and hematoma formation. In an embodiment the initial incision and finger sweep blunt dissection commence in the proper tissue planes in order to minimize any soft tissue injuries. Once the proper plane is entered, dissection should proceed with ease. Dissection into the presacral space 15 should not be forced. As dissection is carried out further in the cephalad direction, a thick fascial layer (Waldeyer's fascia 18) at the level of S3 or S4 may be encountered, requiring more diligent dissection. Again in one embodiment, dissection should be wide enough to accommodate an index finger. Once the presacral space 15 is entered, loose areolar tissue between the presacral fascia and the fascia propria is encountered. The rectum 10 can be gently swept away from the anterior surface of the sacrum 11. At the conclusion of the blunt finger dissection, the rectum 10 may be completely free from the sacrum 11. Adequate dissection can allow the rectum 10 to fall away from the sacrum 11 and potential harm's way.

It should be appreciated that while the finger dissection steps described above are presently preferred it is anticipated that in other embodiments the finger dissection steps described above can be modified, reduced and/or omitted from the procedures and techniques described herein.

U.S. Pat. No. 6,575,979, issued Jun. 10, 2003, titled METHOD AND APPARATUS FOR PROVIDING POSTERIOR OR ANTERIOR TRANS-SACRAL ACCESS TO SPINAL VERTEBRAE, hereby incorporated in its entirety into this disclosure by reference, discloses in detail tools and methodology for accessing targeted treatment sites, such as, for example, inter-vertebral motion segments. Certain of the access and preparation surgical tools, as explained in U.S. Pat. No. 6,575,979, take the form of elongated solid body members extending from proximal to distal ends thereof. As used herein, the term “guide pin” can include solid body members (e.g., guidewires) employed to perform the functions of guide pin delivery and guidance described herein, unless the exclusive use of a given one of such solid body members is explicitly stated. Such solid body members can be stiff or flexible and can include distal anchoring mechanisms, e.g., sharpened or beveled tips. Certain others of the surgical tools take the form of hollow body, tubular members having lumens extending from proximal to distal ends thereof. Such hollow body, tubular members can take the form of medical catheters, medical cannulas, medical tubes, hollow needles, trocars, sheaths, or the like, or variations thereof. Such hollow body tubular members employed in various embodiments described herein can be stiff or flexible and can include distal fixation mechanisms. As used herein, anterior refers to in front of the spinal column (ventral) and posterior refers to behind the column (dorsal). As used herein, proximal (caudal) refers the end or region that is closer to the surgeon or sacral region of the spine, while distal (cephalad) refers to the end or region that is closer to the patient's head. Certain of the surgical tools described herein can include atraumatic and/or malleable distal ends or tips.

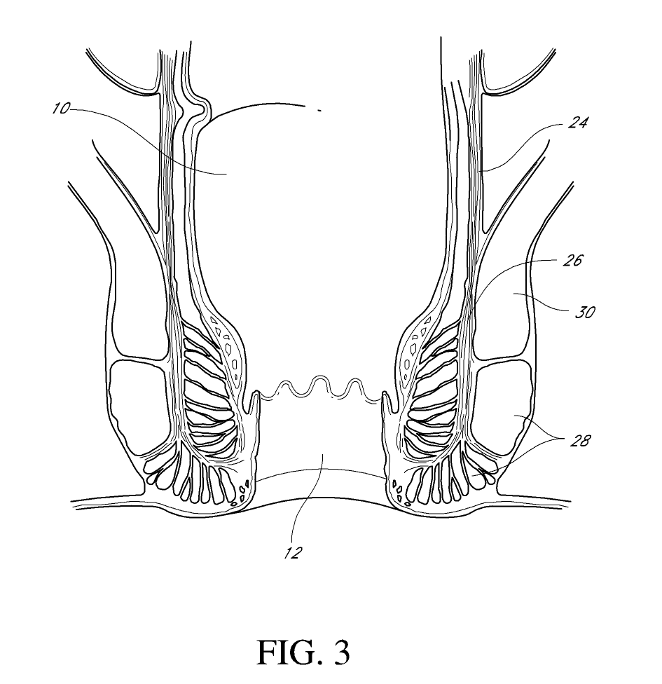

In accordance with one aspect of the embodiments described in U.S. patent application Ser. No. 10/972,065, filed on Oct. 22, 2004 and incorporated by reference, in its entirety, herein, there is provided a guide pin introducer (illustrated in embodiments in at least FIGS. 2A-3E of U.S. patent application Ser. No. 10/972,065) that can be used to facilitate access to the sacrum for delivery of at least one guide pin, which in turn serves as means over which other instruments of the surgical tools set can subsequently be delivered to target sites to perform their intended procedural functions, individually or in combination, over or through one another.

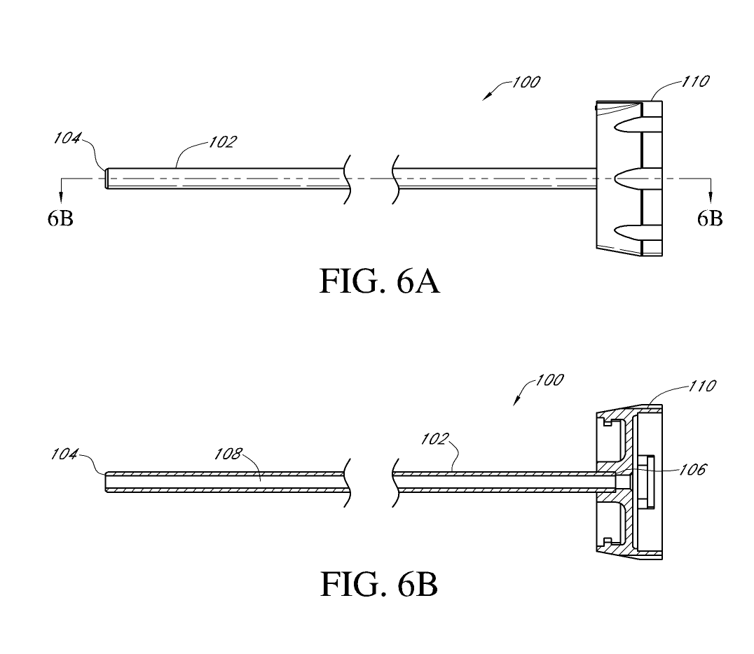

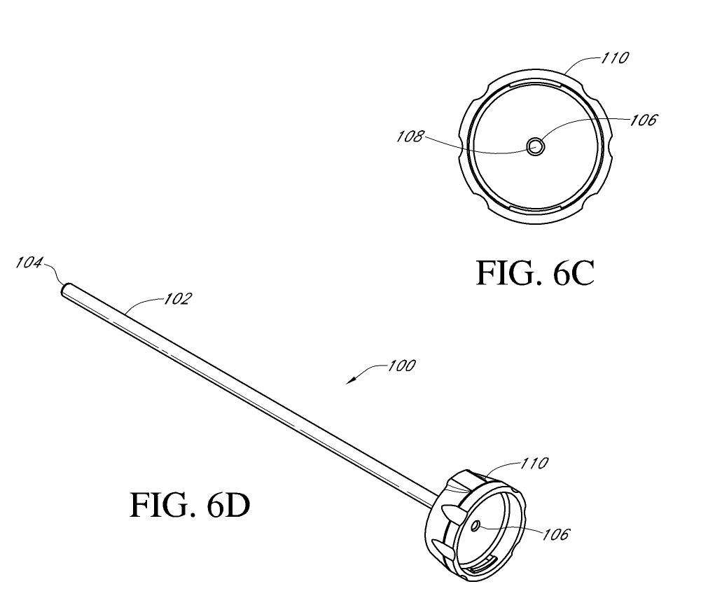

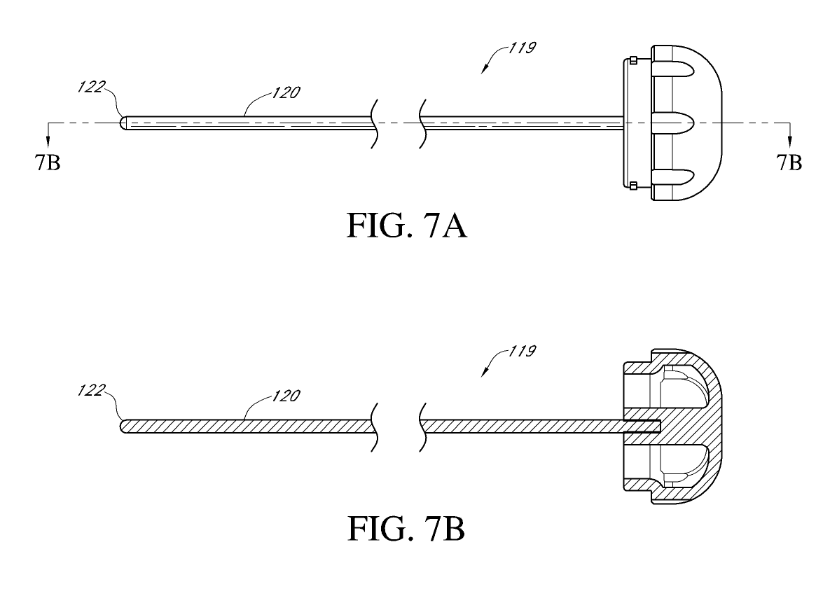

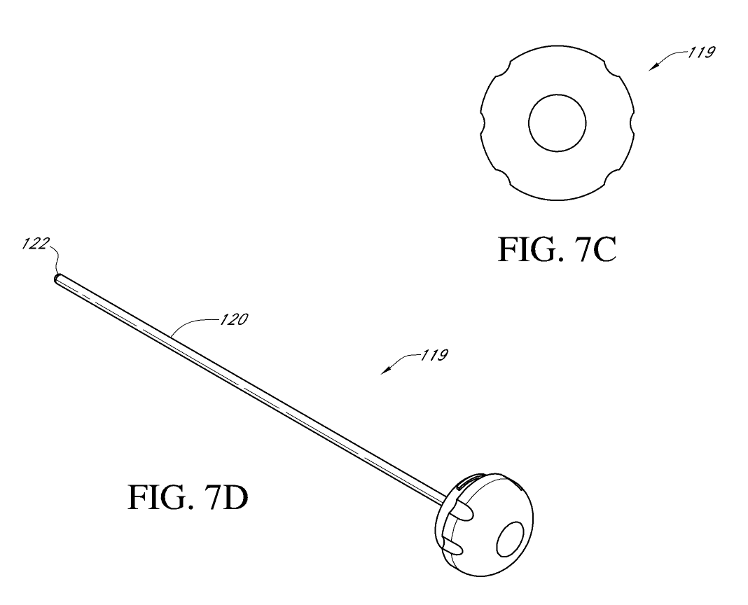

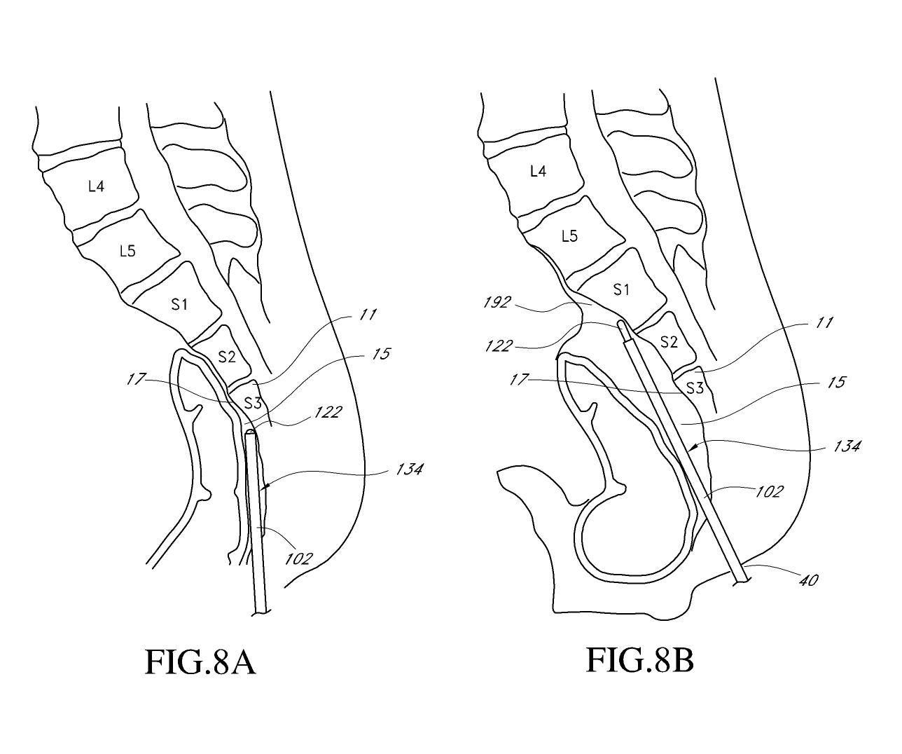

In an embodiment, an access assembly comprises a guide pin introducer 100, as illustrated in FIGS. 6A-D, and a stylet 119, as illustrated in FIGS. 7A-D. The guide pin introducer 100 comprises an introducer tube 102 and an introducer handle 110. The introducer tube 102 extends between a distal end 104 and a proximal end 106, and defines an inner, tubular member lumen 108. The length of the tube 102 is typically in the range of about 4″ (100 mm) to about 12″ (310 mm), often about 5″ (120 mm) to about 9″ (230 mm). In one exemplary embodiment, the length of the tube 102 is approximately 7″ and in other embodiments is approximately 10″. The inner diameter (I.D.) of the introducer tube 102 is typically in the range of about 2 mm to about 5 mm, often about 3 mm to about 4 mm. In an embodiment, the I.D. of the tube 102 is about 3.5 mm (0.13″). The outer diameter (O.D.) of the tube 102 is typically in the range of about 4 mm to about 7 mm, often about 5 mm to about 6 mm. In an embodiment, the O.D. of the tube 102 is about 5.5 mm. In some embodiments, the I.D. is dimensioned to slidably receive the distally located blunt tip 122 on the rod 120 of the stylet 119 illustrated in FIGS. 7A-D. In preferred embodiments, the length of the rod 120 is longer than the length of the tube 102 so that the blunt tip 122 of the rod 120 extends beyond the distal end 104 of the tube 102 when the guide pin introducer 100 and stylet are assembled. The tube 102 and rod 120 are preferably long enough to extend from a skin incision 40 near the paracoccygeal region, through the presacral space 15, to an anterior target site 192, as shown, for example, in FIGS. 8A and 8B. It will be noted that the actual dimensions (e.g., length, inner diameter, outer diameter, etc.) of the guide pin introducer 100 and stylet 119, or any of the tools and components parts thereof described herein will depend in part on the nature of the treatment procedure and the physical characteristics of the patient, as well as the construction materials and intended functionality, as will be apparent to those of skill in the art.

With continued reference to FIGS. 8A and 8B, in one mode of use, the guide pin introducer tube 102 serves as an enlarged diameter anterior tract sheath through which a guide pin can be introduced into the targeted site 192. In a method of use, the spine is accessed via a small skin puncture 40 adjacent to the tip of the coccyx 16. In some embodiments the presacral space 15 can be entered using any known suitable percutaneous technique. The introducer assembly 134, with the stylet's blunt tip 122 serving as a dilator, can be advanced through the paracoccygeal entry site. Once the tip 122 of the stylet 119 is advanced through the fascial layer, the blunt tip 122 can be positioned against the anterior surface 17 of the sacrum 11 and advanced along the anterior surface 17 of the sacrum 11 to the desired position or targeted site 192. In one embodiment, the distal portion of the introducer assembly 134 is advanced to the targeted site under fluoroscopic guidance, as is described in U.S. patent application Ser. No. 10/125,771 filed on Apr. 18, 2002, now U.S. Pat. No. 6,899,716, which is incorporated by reference in its entirety herein.

The stylet 119 can be released and removed from the introducer assembly 134 after the distal portion of the introducer assembly 134 is advanced to the targeted site 192, thereby leaving the distal portion of the introducer 100 at the targeted site, to preface the introduction of a guide pin through the introducer 100 to the targeted site 192. In another embodiment described in U.S. patent application Ser. No. 10/972,065, filed on Oct. 22, 2004 and incorporated by reference, in its entirety, herein, there is a guide pin assembly with an elongate guide pin that extends between a distal end and a proximal end. In one embodiment the guide pin assembly also has a sharp guide pin tip at the distal end and an optional releasable handle engaged at the proximal end.

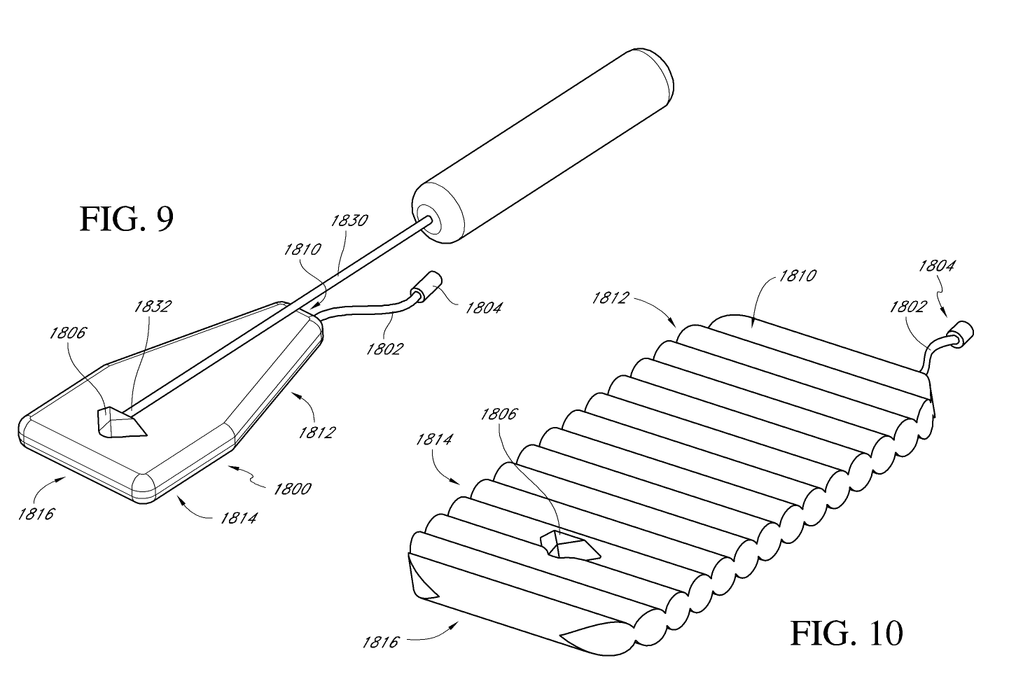

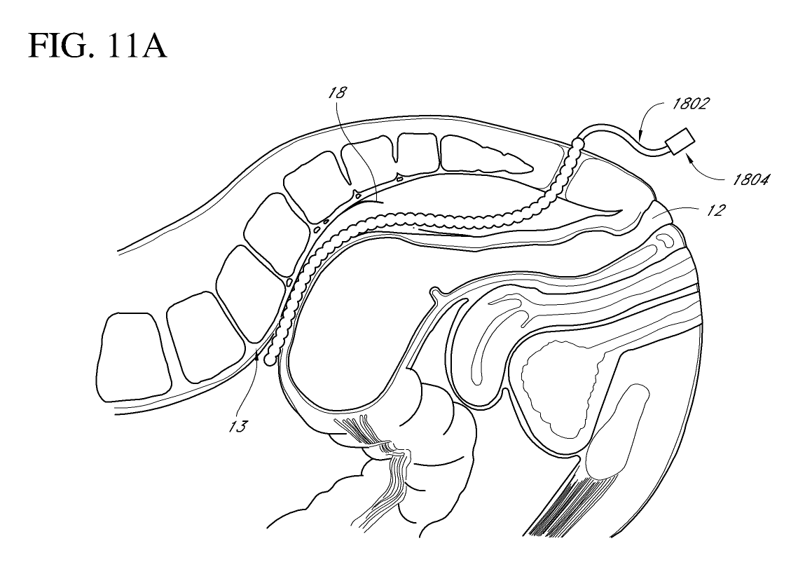

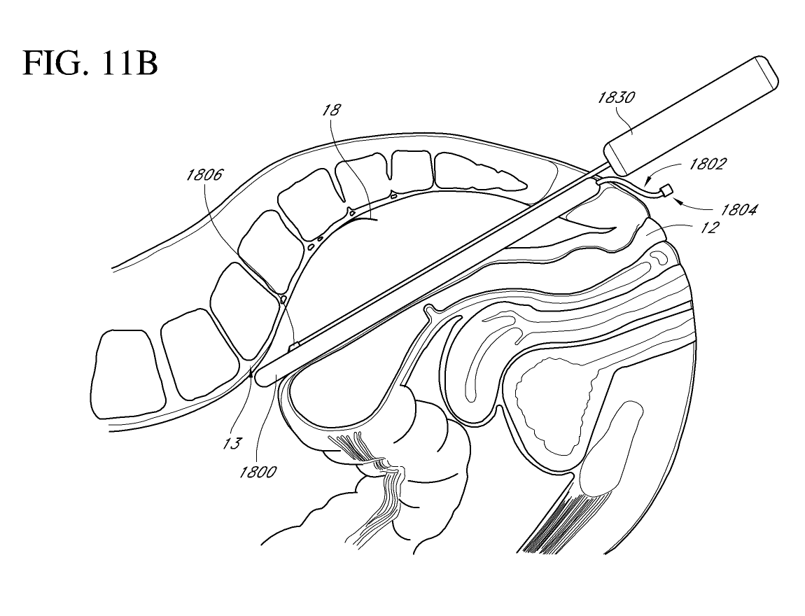

As noted above with respect to FIG. 1, some embodiments can include a tissue retractor that can be inflated or otherwise expanded to define a working space or channel that is generally positioned between the bowel and the sacrum. The working space or channel created by the tissue retraction device 1000 can form a portal that extends from a target site (e.g., on a patient's sacrum) towards or to an surgical access site such that tools and instruments can be inserted from the surgical access site, through the portal and to the target site. FIGS. 9 and 10, illustrate one embodiment of a tissue retraction device 1800 for retracting the soft tissue in the presacral space 15. In this embodiment, the tissue retractor 1800 is configured to retract the bowel from the sacrum and to protect all tissue from subsequent instrumentation movement (insertion, actuation, removal) in the space. In various embodiments, the tissue retractor 1800 can be deployed along any lengths or varying heights or depths into the retrorectal or presacral space 15, as illustrated in FIG. 11A. In some embodiments, the tissue retractor 1800 can comprise a generally flat, shaped structure that can be inserted into the presacral space 15 and then inflated to retract the bowel from the sacrum 11. The flat structure advantageously provides a wide surface to abut or butt against the sacrum 11 on one side and retract the bowel or other soft tissue on the other side. In a preferred embodiment, the width of the tissue retractor 1800, measured as the dimension that extends substantially perpendicular to the proximal-distal orientation of the tissue retractor 1800, is approximately 5-8 centimeters. In some embodiments, the width of the tissue retractor 1800 ranges from about 2.5 centimeters to 16 centimeters. Preferably, the surfaces of the tissue retractor 1800 are compliant and can conform to the native anatomy. In a preferred embodiment, the tissue retractor 1800 is made of polyurethane. In other embodiments, the tissue retractor 1800 can be made of any pliable material that can be folded or rolled, such as for example silicone, elastomer, polyethylene or Teflon®. The non-rigid surfaces can also avoid injury to the soft tissue in the presacral space 15 by the tissue retractor 1800.

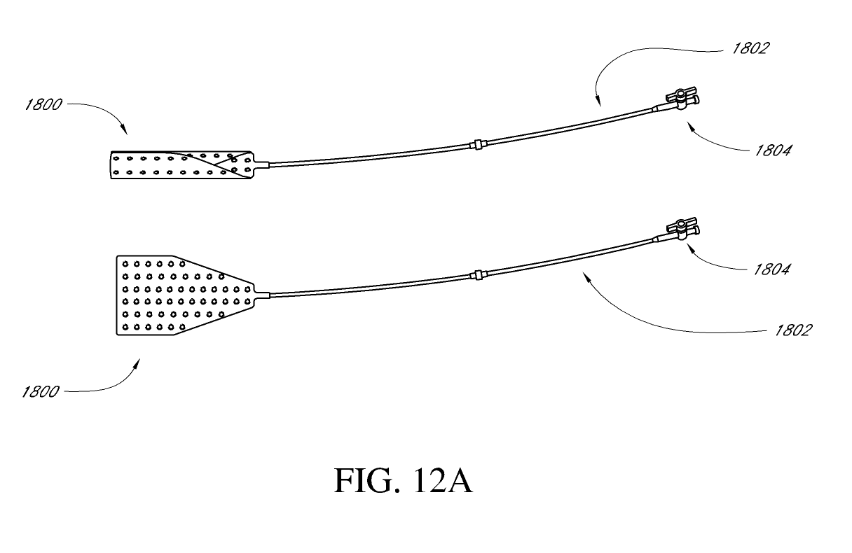

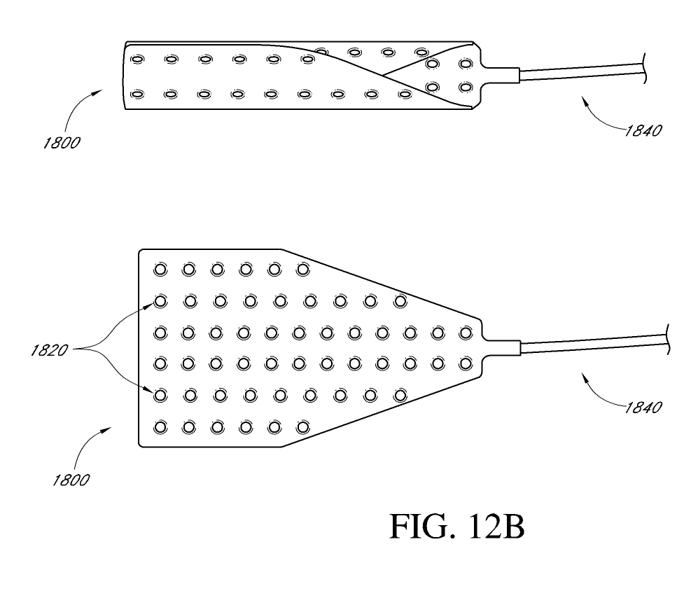

In some embodiments, the tissue retractor 1800 can have top and bottom layers that are connected together at discrete tack joints 1820 to form an array, such as the “pillow-top” surface illustrated in the embodiment of FIGS. 12A-B. The thickness of the tissue retractor 1800 in the inflated configuration can be restricted by the joints 1820. Some embodiments of the tissue retractor 1800 can be configured to expand to varying thicknesses to vary the retraction of the tissue at different locations, determined by the joints 1820 located at the different sections. In some embodiments, the surfaces of the tissue retractor 1800 are baffled or corrugated. The tissue retractor 1800 can be baffled or corrugated in a proximal-distal orientation. In other embodiments, as illustrated in FIG. 10, the tissue retractor 1800 is baffled or corrugated in a lateral orientation, substantially perpendicular or normal to the proximal-distal orientation. In various embodiments the tissue retractor 1800 is baffled or corrugated in any angles (e.g., along the longitudinal axis in a distal to proximal direction), sloped, curved, or other orientation. However, in some embodiments, the flexible surfaces are substantially flat or planar.

In some embodiments, the tissue retractor 1800 comprises a proximal end 1810, proximal portion 1812, distal portion 1814, and a distal end 1816. In various embodiments, the tissue retractor 1800 can have various shapes, such as a rectangle as illustrated in FIG. 10, a triangular shape, oval shape, or rounded shape. In yet other embodiments (described below), the retractor can define also define an opening or channel extending from the distal end to the proximal end of the device and/or can have a cylindrical, tubular shape that defines a elongate opening through the device. In some embodiments, such as illustrated in FIGS. 12A and 12B, the proximal portion 1812 and distal portion 1814 can have different dimensions. The width of the tissue retractor 1800 can be wider at the distal portion 1814 to enable retraction of more of the bowel adjacent the target site 192. In an embodiment illustrated in FIGS. 12A and 12B, the width at the distal portion 1814 can range from about 5 centimeters to about 8 centimeters. The width at the proximal portion 1812 can be about 2.5 centimeters. In the illustrated embodiment, the thickness of the tissue retractor 1800 is approximately 1 centimeter. In other embodiments, the thickness can range from about 0.5 centimeter to about 2.5 centimeters. The length, measured as the proximal-distal dimension, is preferably approximately 13 centimeters. In other embodiments, the length can range from about 5 centimeters to about 26 centimeters. In still other embodiments, the thickness, width and length can be any dimension appropriate for a patient's anatomy.

In some embodiments, the tissue retractor 1800 can be coupled to an inserter 1830 for insertion into the presacral space 15, as illustrated in FIG. 9. In the illustrated embodiment, the inserter 1830 is an elongate rod with a handle at the proximal end and a distal end 1832 coupled to the tissue retractor 1800. In some embodiments, the inserter 1830 can be any tool capable of introducing and/or positioning the tissue retractor 1800 into the presacral space 15, such as for example an introducer, a guide pin introducer, a pushrod, a dissecting tool, etc. In an embodiment, the tissue retractor 1800 can comprise an interface 1806 within which the distal end 1832 of the inserter 1830 can be inserted. In various embodiments, the interface 1806 can be a pocket, a link, a releasable lock, a permanent lock, or other connection. In the illustrated embodiment, the interface 1806 is positioned on the top of the tissue retractor 1800 however, in other embodiments; the interface 1806 can be positioned on the bottom of the retractor 1800 and/or on a side of the retractor 1800. Once the tissue retractor 1800 is in place, the inserter 1830 can be removed while the tissue retractor 1800 remains in situ. In some embodiments, the inserter 1830 can remain anchored to the tissue retractor 1800 after placement and inflation to allow for manipulation of the tissue retractor 1800 during the surgical procedure.

In some embodiments, the bowel is dissected from the sacrum 11 (e.g., using the finger dissection and/or guide pin techniques described above) and then the tissue retractor 1800 is introduced into the presacral space 15. In other embodiments, the tissue retractor 1800 can be coupled to the inserter 1830 and the bowel can be dissected from the sacrum 11 at the same time that the tissue retractor 1800 is introduced into the presacral space 15. Dissection can also occur as the tissue retractor 1800 is inflated as described below. In the uninflated configuration, the tissue retractor 1800 can be yielding and can be inserted easily through the skin incision.

In a preferred embodiment, the tissue retractor 1800 can be compacted by folding or rolling the sides of the tissue retractor 1800 along the proximal distal length, such as illustrated in FIGS. 12A and 12B. The folded/rolled configuration allows for easier insertion through the incision 40 and into the presacral space 15. In an embodiment, a sheath surrounds the folded or rolled tissue retractor 1800 to hold it in the folded/rolled configuration. The sheath can be configured to release the tissue retractor 1800 when the tissue retractor 1800 is inflated. For example, the sheath can comprise a perforated line along which the sheath breaks open as the tissue retractor 1800 is inflated. In an embodiment, the sheath and tissue retractor 1800 remain attached. In an embodiment, while the sheath opens along the perforated line it remains attached to the tissue retractor 1800. In another embodiment, the sheath can comprise a latch that disengages when a predetermined tensile force is applied to the latch by the inflating tissue retractor 1800. The sheath is preferably secured to the tissue retractor 1800 to aid in the recovery and removal of the sheath from the surgical site. In other embodiments, the tissue retractor 1800 can be held in the rolled configuration by a tack joint connecting the sides of the tissue retractor 1800. When the tissue retractor 1800 is inflated, the tack joint can release, allowing the tissue retractor 1800 to unfold inside the presacral space 15. In other embodiments, the tissue retractor 1800 can be held in the rolled configuration through other means that will release once the tissue retractor 1800 is inflated. In an embodiment, the tissue retractor 1800 is rolled or folded into a cylindrical configuration having a width (e.g. diameter) of approximately ½ inch to 1 inch (about 1.25 centimeters to 2.54 centimeters). In some embodiments, the tissue retractor 1800 has a length of between about 3 and 7 inches (about 7.6 centimeters-18 centimeters). In one embodiment, the tissue retractor 1800 has a length of 5 inches (about 12.7 centimeters). In some embodiments, the tissue retractor 1800 can be folded into other configurations, such that the width of the tissue retractor is preferably about ½ inch (about 1.1 centimeters to 1.3 centimeters). In an embodiment, the sheath and/or tissue retractor 1800 is coated with a surfactant to assist in the insertion and placement of the tissue retractor 1800. For example, in one embodiment, a biocompatible medical coating or surfactant can be applied to the sheath, inserter, and/or tissue retractor 1800 to enhance hydrophilicity and reduce friction between the tissue retractor 1800 and the native anatomy.

In a preferred embodiment, the tissue retractor 1800 is positioned to extend from at least the target site 192 to the incision 40, as illustrated in FIG. 11B. In some embodiments, the tissue retractor 1800 is positioned toward the distal portion of the presacral space 15, near the target site 192. For example, the tissue retractor 1800 can be positioned adjacent the sacrum 11 between the top of the target site 192 and the sacral promontory 13. In other embodiments, the tissue retractor 1800 can be positioned over other portions within the presacral space 15.

In an embodiment, the tissue retractor 1800 can include an inflation tube 1802 and a port 1804 that can preferably extend outside of the patient's body for connecting to an inflation media source. Preferably, the port 1804 includes a valve to close the port once the inflation media is inserted and also to open the port when the tissue retractor 1800 is to be deflated. It should be appreciated, that in other or modified embodiments of the tissue retractor described herein, the valve can be positioned within the inflation tube closer to the tissue retractor and/or within the tissue retractor.

Once the tissue retractor 1800 is positioned inside the presacral space 15, the tissue retractor 1800 can be filled with an inflation media to deploy the tissue retractor 1800 into the inflated configuration. In some embodiments, contrast media that is radio opaque can be used for visualization with fluoroscopy during computed tomography (CT) or X-ray imaging. An example of contrast media is called Hypaque™. In some embodiments, the tissue retractor 1800 can be filled with saline. Preferably, some contrast media is mixed with the saline to allow the tissue retractor 1800 to be visible, but still transparent enough to allow other instruments to also be visualized through the tissue retractor 1800. In a preferred embodiment, the mixture can comprise of a percentage of Hypaque and a percentage of saline. In other embodiments, the tissue retractor 1800 can be inflated with air. In other embodiments, the tissue retractor can be filled with other inflation media, such as water, nitrogen, foam, or any other suitable inflation media. Preferably the inflation media is biocompatible in case of rupture of the tissue retractor 1800 and leakage of the inflation media into the presacral space 15. Inflation media is thus a broad terming that includes liquids, gasses, gels, and foams.

In some embodiments, the tissue retractor 1800 can include more than one inflation chamber. For example, a first inflation chamber can be disposed on the left side of the tissue retractor 1800 and a second inflation chamber can be disposed on the right side of the tissue retractor 1800, such that the junction between the first and second inflation chambers extends in the proximal-distal direction. The separate inflation chambers can allow the surgeon to inflate each side separately for more control during retraction, particularly in situations that require asymmetric retraction. Similarly, in another embodiment, multiple inflation chambers can be disposed from the distal portion to the proximal portion of the tissue retractor 1800 such that the distal portion can be inflated separately from the proximal portion. In yet another embodiment, the multiple (i.e., more than one) inflation chambers can be positioned from a superior portion (upper) to an inferior portion (lower) of the retractor 1800. In addition various combinations and sub-combinations of upper, lower, front, back, left, and inflation chambers can be used. Multiple inflation chambers advantageously allow for staged inflation where the different inflation chambers can be inflated in a particular sequence for maximum effective retraction of the bowel. In addition, multiple inflation chambers can help limit the amount of leakage of the inflation media in case of rupture. In certain embodiments, uninflated portions of the retractor can be positioned between inflation chambers. For example, one embodiment an uninflated member can extend between two inflation chambers such that the uninflated member defines part of the barrier formed by the retractor.

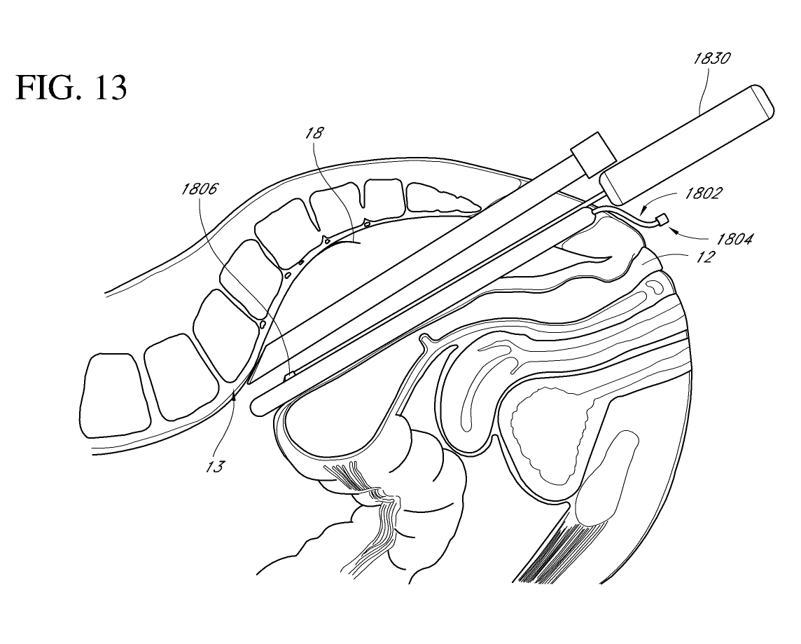

A goal of some embodiments of the tissue retractor 1800 is to keep the bowel away from the anterior surface 17 and to protect and/or preclude instrumentation that is inserted during the surgical procedure from touching the bowel. In other words, the tissue retractor 1800 retracts the bowel away from the anterior surface 17 to create a working channel for instrumentation to pass without obstruction from the bowel. As illustrated in FIG. 13, the surgical devices and methods, as described herein, can be used within the working channel created by the tissue retractor 1800. Risk of inadvertent injury to the bowel can be minimized through the use of the tissue retractor 1800 because devices can be inserted to the target site 192 and removed from the presacral space 15 without contact with the bowel. In certain embodiments, the tissue retractor 1800 provides 90° to 120° of protection for the working channel. In some embodiments, the tissue retractor can provide protection that covers less than or equal to 180° of the working channel. For example, in the embodiment of FIG. 13, the retractor 1800 can provide protection equal to about 180° as the retractor 1800 is positioned generally between the bowel and the sacrum with a working channel being defined generally above the retractor 1800, which forms a barrier between tools inserted into the working channel and the bowel. As will be described in detail below, further embodiments can provide greater than 180° of protection.

After the surgical methods are performed through the presacral space 15 and the instrumentation is removed, the tissue retractor 1800 can be removed. In one embodiment, removal of the tissue retractor 1800 includes deflating the tissue retractor 1800 by opening the port 1804 on the inflation tube 1802. In some embodiments, the inflation media can be removed by applying a negative pressure at the port 1804 to suck the inflation media out of the tissue retractor 1800. In some embodiments, the inserter 1830 or some other device can be used to compress the tissue retractor 1800 and squeeze the inflation media out of the tissue retractor 1800. Once the tissue retractor 1800 is sufficiently deflated and compliant to exit through the incision 40, the tissue retractor 1800 can be extracted by being held at the proximal end and pulled through the incision 40. In other embodiments, the tissue retractor 1800 can be pulled by the inflation tube 1802 or port 1804. In some embodiments, the tissue retractor 1800 can be predisposed to return to the rolled or folded configuration when deflated for easier removal through the incision 40. In other embodiments wherein the inserter 1830 is fixed to the tissue retractor 1800, the inserter 1830 can be pulled out of the incision 40 to remove the attached tissue retractor 1800. In another arrangement, the inserter 1830 can coupled to the tissue retractor 1800 (after being previously removed) and then used to withdraw the tissue retractor 1800 from the patient.

In one embodiment, removal of a tissue retractor 1800 includes drawing media from the tissue retractor 1800 with relatively low pressure or a vacuum. In one embodiment, a syringe or low pressure source can be used to remove media from the tissue retractor 1800. The deflation of the tissue retractor 1800 can be confirmed through visualization, such as fluoroscopy. In various embodiments, the tissue retractor 1800 can be withdrawn with the inflation tube 1802 or an inserter 1830, or both. In one embodiment, the tissue retractor 1800 can be re-sheathed before withdrawal or removal, but in another embodiment, the tissue retractor 1800 is simply withdrawn once deflated.

In one example embodiment of use of the retractor embodiments described above, the surgeon, following a finger sweep dissection technique, dissects further using a dissector (which may or may not have the tissue retractor 1800 attached to it). The surgeon can then dissect up to the sacral promontory with the dissector. If the tissue retractor 1800 is not attached to dissector, the tissue retractor 1800 can be inserted in the space created by the dissector using the introducer to position the tissue retractor 1800. Once the tissue retractor 1800 is in position as confirmed under fluoroscopy, a contrast/saline mixture can injected into the retractor 1800 slowly under live fluoroscopy using syringe. Once properly filled, a fluid flow control valve (e.g., a stopcock) can be turned into a closed position to maintain the retractor in an inflated condition and the syringe can be detached. With the bowel retracted away from the sacrum, tools can be inserted into the protected pre-sacral space with the retractor 1800 acting as a barrier between the tools and the bowel as the tools are advance toward a target site on the sacrum. In one embodiment, the spinal techniques described above (e.g., AxiaLIF® Procedures) can be performed on the spine with the tools (e.g., guidewire, bone dilators, drills, implant drivers, tissue cutters and extraction devices) inserted into the protected pre-sacral space created by the retractor 1800. After the procedure is completed, an empty syringe can be attached to a luer fitting provided on the retractor 1800 to draw vacuum on the tissue retractor 1800 with the syringe under real-time fluoroscopy. The surgeon can confirm with fluoroscopy that the retractor 1800 is deflated and then can withdraw the retactor 1800 using the inflation tube or the introducer/inserter (if an inserter attached). In one arrangement, the retractor 1800 can be repositioned after it is initially expanded. Repositioning can occur while the device is partially expanded, fully expanded, and/or after it has been partially deflated or otherwise reduced in diameter.

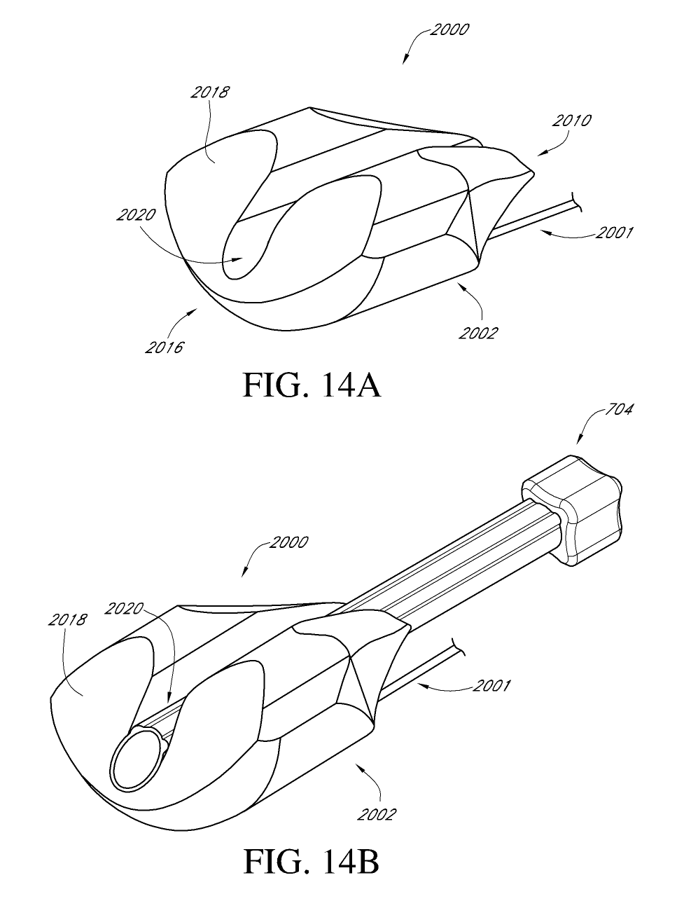

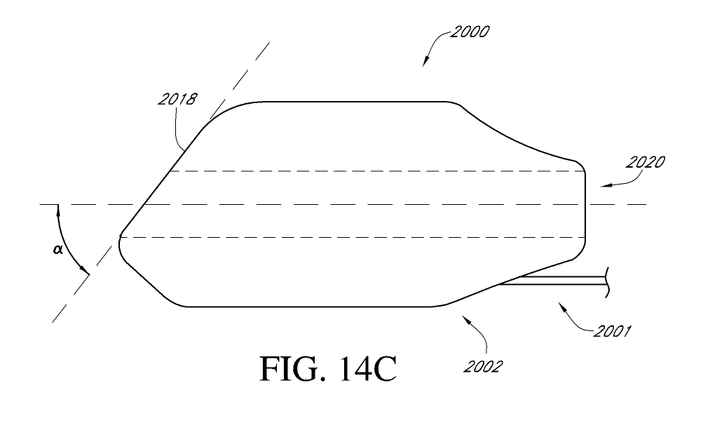

In other embodiments of surgical kits or assemblies, a tissue retractor 2000 can at least partially form a channel 2020 that extends through at least a portion of an inflatable body 2002, as illustrated in FIGS. 14A-B and 15A-B. As described above in other embodiments, the tissue retractor 2000 can be configured to retract bowel from the sacrum 11 and can be configured to maintain the access to the target site 192 and/or protect the tissue from subsequent instrumentation movement (insertion, actuation, removal) in the space. The channel 2020 can extend from the proximal end 2010 to the distal end 2016 of the tissue retractor 2000 to provide a pathway for surgical tools.

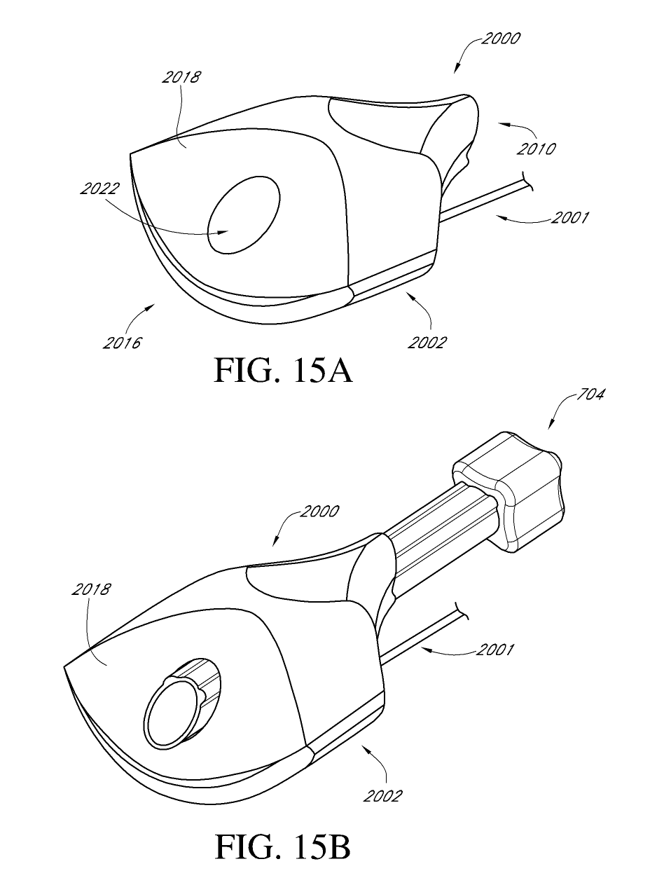

In some embodiments, a tubular retractor 704 can be inserted through the channel 2020 to create a working channel for a surgical procedure, as illustrated in FIGS. 14B and 15B. In preferred embodiments, the tubular retractor 704 can have a distal tip that can conform to the contours of the surface of the sacrum 11. The distal tip can be made of a soft and/or compliant material, such as for example rubber or silicone. In some embodiments, the distal tip can include an inflatable balloon (not shown) that can conform to the surface of the sacrum 11. A plurality of other types of materials can be used on the compliant distal tip of the tubular retractor 704. In one embodiment, the tubular retractor 704 can include one or more longitudinal pathways (not shown) that extend from a proximal end of the device and emerge at the distal end of the device extending from the tapered front surface. Fixation wires with threaded distal ends can be inserted through the one or more pathways to secure the tubular retractor 704 to the sacrum.

The tissue retractor 2000 can comprise an inflatable body 2002 that is contoured to form a channel 2020 when inflated. The inflatable body 2002 can be inflated through an inflation tube 2001 that is in fluid communication with the inner chamber of the inflatable body 2002. Preferably, the inflation tube 2001 includes a valve to seal the inflatable body 2002. The channel 2020 can extend generally through the middle of the inflated body 2002, such as illustrated in the embodiments of FIGS. 14 and 15. In the embodiment illustrated in FIG. 14A, the channel 2020 is open on one side, such that the channel 2020 has a cross-section that is generally U-shaped. The open channel advantageously allows placement of devices in the channel 2020 from a lateral direction, and does not limit the placement of devices within the channel 2020 through the proximal end 2010 or distal end 2016 of the retractor 2000. In some embodiments, as illustrated in FIG. 15A, the channel 2022 can be enclosed and can be accessed from the proximal end 2010 and distal end 2016 of the retractor. The enclosed channel 2022 can advantageously be surrounded by the inflatable body 2002 to shield the pathway from portions of the native anatomy. In some embodiments, as illustrated in FIGS. 14B and 15B, an tubular retractor 704 can be inserted into the channel 2020, 2022 to provide a protected working pathway to the target site 192. For example, the tubular retractor 704 can provide a more rigid pathway and protect the inflatable body 2002 from potential punctures by the sharp instruments inserted to the target site 192 through the tubular retractor 700. In certain embodiments, the channel 2020, 2022 has a large enough cross-sectional profile such that the target site at the distal end 2016 of the retractor can be visualized from the proximal end 2010 of the retractor 2000.

In the embodiment of FIG. 15A, the enclosed channel 2022 is defined by inflated portions of the inflatable body 2002. However, in modified embodiments, the enclosed channel can be defined, at least in part, by an uninflated portion of the retractor 2000. For example, in one embodiment, a top or bottom portion of the channel 2022 can be defined by an uninflated flexible or rigid sleeve that is coupled to inflated portions of the retractor 2000. In such embodiments, the sleeve can protect the patient's soft tissues as tools are inserted into the channel 2022. In still other embodiments (or in combination with other embodiments), longitudinal portions (e.g., a distal, proximal or middle portion) of the channel 2022 can be defined, in part, by an uninflated portion of the retractor 2000. For example, in an arrangement, a distal portion of the channel 2022 is defined by inflated portions of the retractor while a proximal portion of the channel 2022 is defined by an uninflated portion (e.g., a flexible or rigid sleeve).

In some embodiments, the width of the tissue retractor 2000, measured as the dimension that extends substantially lateral to the proximal-distal orientation of the tissue retractor 2000, is approximately 4-13 centimeters. In some embodiments, the thickness of the tissue retractor 2000, measured in the direction of the depth of the U-shape of the channel, can ranges from about 2.5 centimeters in some portions to about 16 centimeters in other portions. The length, measured as the proximal-distal dimension, can be approximately 13 centimeters. In other embodiments, the length can range from about 5 centimeters to 26 centimeters. In still other embodiments, the thickness, width and length can be a plurality of different dimension appropriate for a patient's anatomy.

In some embodiments, the tissue retractor 2000 can be made of polyurethane. In other embodiments, the tissue retractor 2000 can be made of any pliable material that can be collapsed and inflated, such as for example silicone, elastomer, polyethylene or Teflon®. The non-rigid surfaces can also avoid injury to the soft tissue in the presacral space 15 by the tissue retractor 2000. Preferably, the surfaces of the tissue retractor 2000 are compliant and can conform to the native anatomy. However, certain embodiments can include less complaint materials.

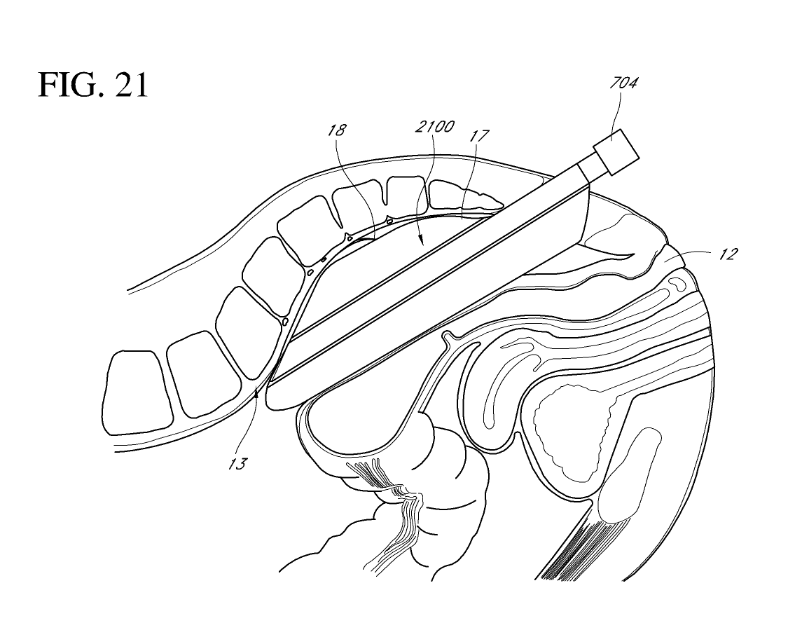

The tissue retractor 2000 can be inserted into the presacral space 15 and then inflated to retract the bowel from the sacrum 11. The body 2002 advantageously provides a surface to abut against the sacrum 11 on at least one side of the tissue retractor 2000 and retract the bowel or other soft tissue on another side (e.g., a bottom side in the illustrated embodiment). In some embodiments, the distal end 2016 of the tissue retractor 2000 can have a tapered, angled or beveled surface 2018, as illustrated in FIGS. 14A-C. With reference to FIG. 14C, the tapered surface 2018 can be at an angle α with respect to the longitudinal, or proximal-distal axis, of the tissue retractor 2000. In preferred embodiments, the angle α is generally the same as the angle of the surface of the sacrum 11 at the target site 192. In some embodiments, the angle α can be approximately 60°. In some embodiments, the angle α can range from at least approximately 25° and/or less than or equal to approximately 75°. The tapered surface 2018 can be generally flat or have a convex shape, such that the tapered surface 2018 can have a generally flush abutment against the sacrum 11, as illustrated in FIG. 21. The tapered surface 2018 can be compliant and conform to the native anatomy of the surface of the sacrum 11. The distal opening of the channel 2020 can be disposed on the tapered surface 2018 to provide access through the tissue retractor 2000 to the sacrum 11. Advantageously, the distal portions (e.g., the tapered surface 2018) of the retractor can also conform to the face of the sacrum when it is pressed against the sacrum to further promote a flush engagement with the sacrum

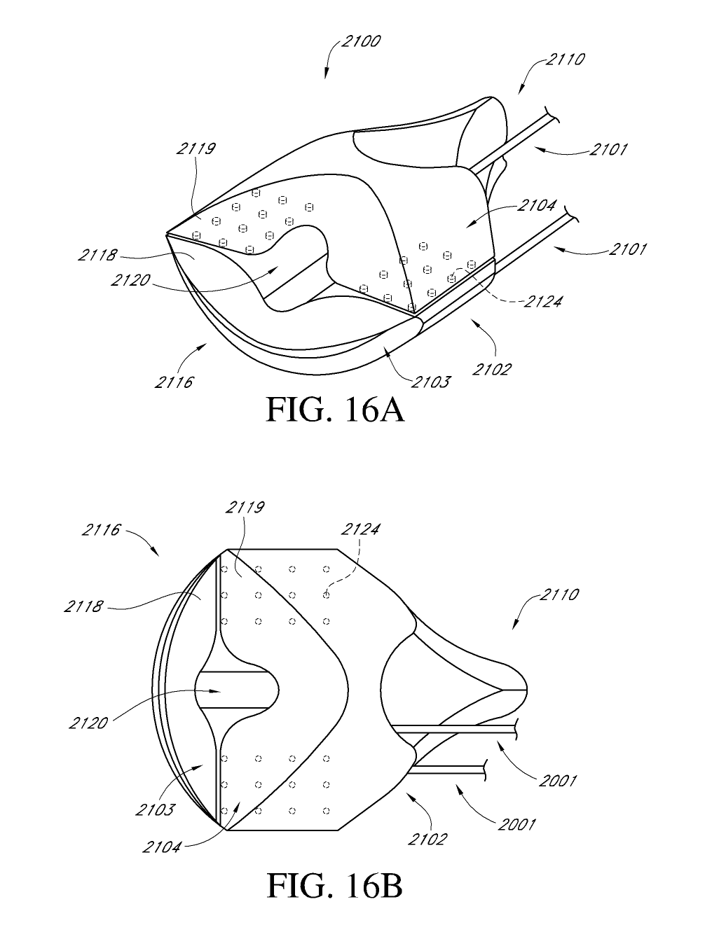

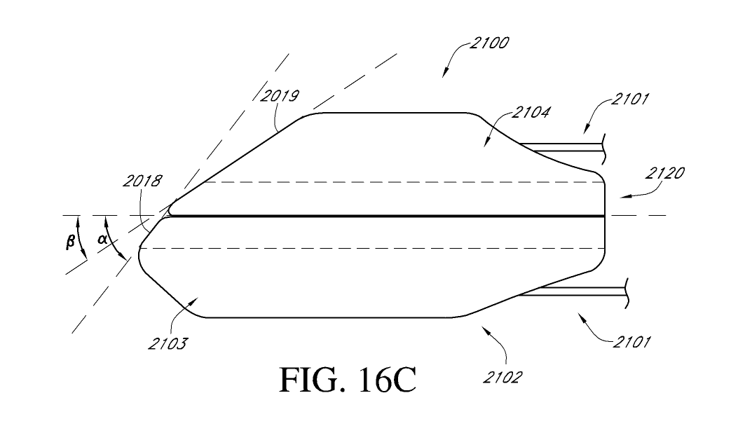

In some embodiments, the tissue retractor 2100 can have a body 2102 with a bottom portion 2103 and a top portion 2104, as illustrated in FIGS. 16A and 16B. The bottom portion 2103 can retract the bowel toward one side and the top portion 2104 can abut against the sacrum 11. The bottom portion 2103 and top portion 2104 can be attached together to form a channel 2120 between the portions. The channel 2120 can be enclosed to provide protection from portions of the native anatomy, as described above in other embodiments. In some embodiments, the bottom portion 2103 and top portion 2104 can be attached by a functional coupler, such as with adhesives, sonic welding, etc. In the illustrated embodiment, the bottom portion 2103 and top portion 2104 are connected together by a plurality of discrete tack joints 2124. In other arrangements, the bottom and top portions can be formed from two halves a balloon welded to a single center sheet at the lateral wings to create two chambers. Extra material can be provided in the center to create the working channel.

The channel 2120 can extend from the proximal end 2110 to the distal end 2116 to provide a working pathway for delivery of devices or tools to the target site 192. In some embodiments, the bottom portion 2103 can be inflated separately from the top portion 2104. The bottom portion 2103 can have an inflation tube 2101 and the top portion 2104 can have a separate inflation tube 2101. The ability to separately inflate the bottom portion and top portion can provide the surgeon better control to customize the tissue retractor 2100 to the particular anatomy of patients, which can allow the surgeon to better position and orient the channel, and consequently more accurately place the tools through the channel 2120. For example, to lower the position of the channel for improved alignment with the target site 192, the surgeon can increase inflation of the top potion 2104, providing more distraction from the sacrum 11.

With continued reference to FIGS. 16A-B, the tissue retractor 2100 can have a first tapered surface 2118 disposed on the distal end 2116 of the bottom portion 2103. A second tapered surface 2119 can be disposed on the distal end 2116 of the top portion 2104. As described above in other embodiments, the first tapered surface 2118 can be at an angle α with respect to the longitudinal axis of the tissue retractor 2100. Similarly, the second tapered surface 2119 can be at an angle β with respect to the longitudinal axis of the tissue retractor 2100. In preferred embodiments, the angles α, β are generally the same as the angle of the surface of the sacrum 11 at the target site 192. In some embodiments, the angle α can be equal to the angle β. In other embodiments, the angles α, β can be different. Having different angles can help the distal end 2116 conform better to the surface of the sacrum 11. In some embodiments, the angle α can be approximately 60°. In some embodiments, the angle α can range from at least approximately 25° and/or less than or equal to approximately 75°. In some embodiments, the angle β can be approximately 30°. In some embodiments, the angle β can range from at least approximately 5° and/or less than or equal to approximately 45°. The first tapered surface 2118 and second tapered surface 2119 can be generally flat or have a convex shape, such that the tapered surfaces 2118, 2119 can have a generally flush abutment against the sacrum 11, as illustrated in FIG. 21. The tapered surfaces 2118, 2119 can be compliant and conform to the native anatomy of the surface of the sacrum 11.

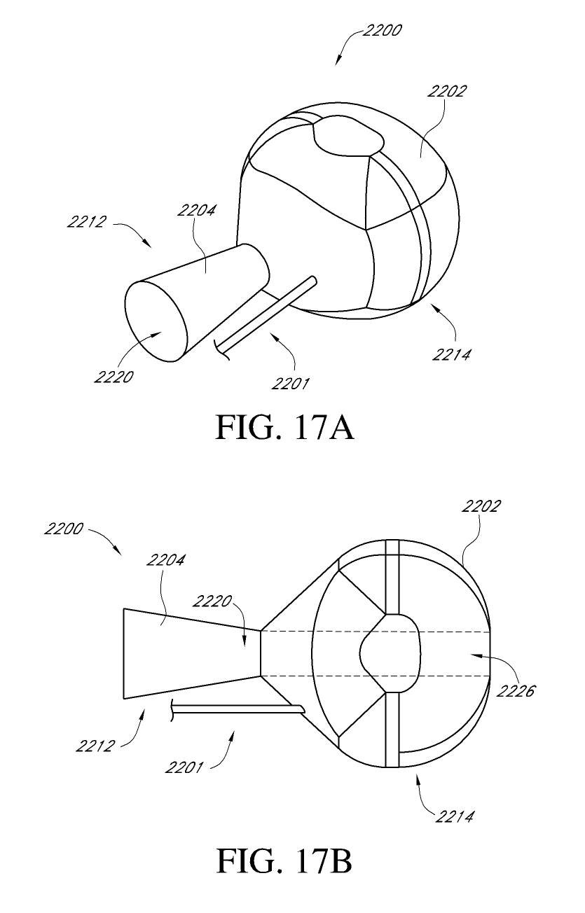

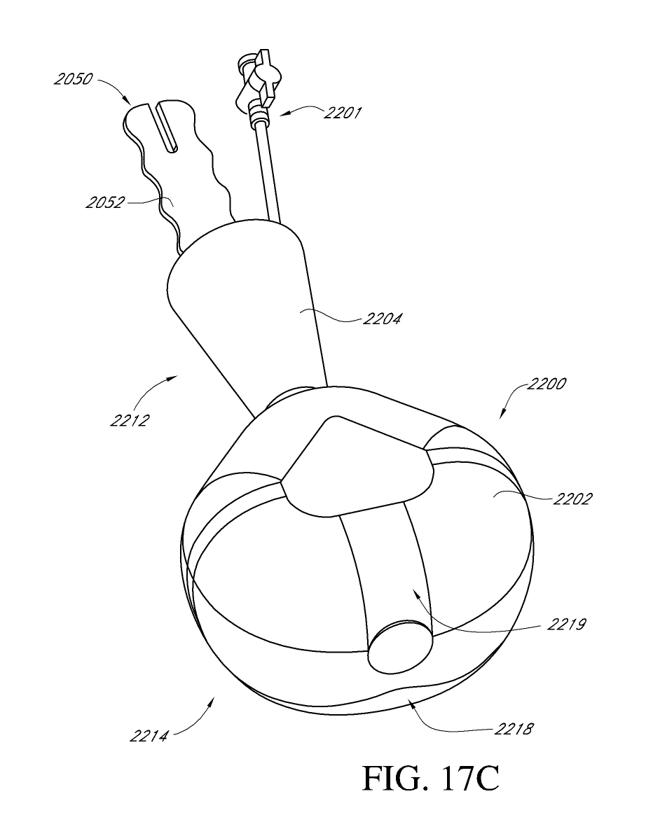

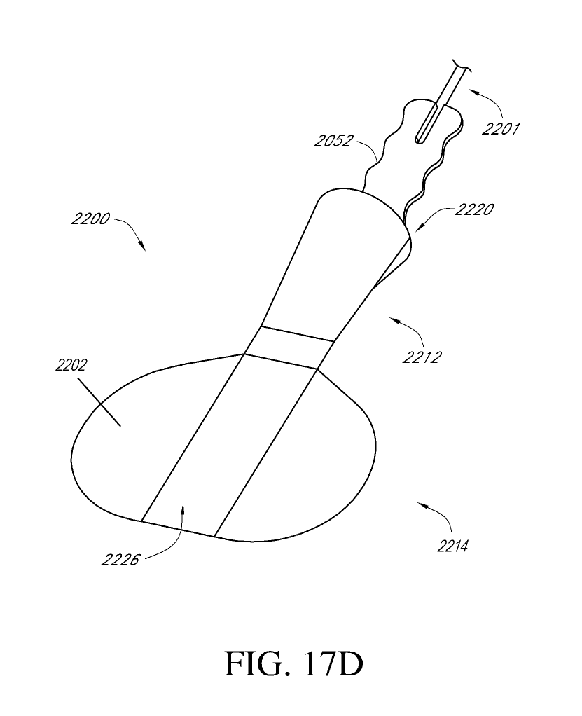

With reference to FIGS. 17A-D, in some embodiments the tissue retractor 2200 can have a proximal portion 2212 and a distal portion 2214. In the illustrated embodiment, the distal portion 2214 is an inflatable body 2202 that is in fluid communication with an inflation tube 2201, and the proximal portion 2212 is a funnel 2204 that, in some embodiments, is not inflated. The funnel 2204 can have a frustoconical shape wherein the proximal end has a larger circumference than the distal end. An o-ring or other support device can be attached to the proximal end of the funnel 2204 to help maintain the opening to the channel 2220. The inflatable body 2202 can have a lumen 2226 that extends from the distal end of the proximal portion 2212 to the distal end of the inflatable body 2202, as illustrated in FIG. 17B. The lumen 2226 can be in fluid communication with the funnel 2204 wherein a channel 2220 can be formed that extends from the proximal end of the funnel 2204 to the distal end of the inflatable body 2202. As noted above, “fluid” as used herein is a broad term that includes liquids, gases (e.g., air), foams, and gels.

With reference to FIG. 17C, the distal end of the inflatable body 2202 can have a bottom portion 2218 and a top portion 2219. In some embodiments, the distal end of the inflatable body 2202 can be configured such that the bottom portion 2218 extends distally further than the top portion 2219, providing a sloped shape at the distal end. For example, the bottom portion 2218 can be contoured to bulge out further than the top portion 2219. The distal end can have an angle α with respect to the longitudinal axis of the tissue retractor 2200. In preferred embodiments, the angle α is generally the same as the angle of the surface of the sacrum 11 at the target site 192 to conform better to the surface of the sacrum 11. In some embodiments, the angle α can be approximately 60°. In some embodiments, the angle α can range from at least approximately 0° and/or less than or equal to approximately 90°. In the illustrated embodiment, the bottom portion 2218 and top portion 2219 have a convex shape that can abut generally flush against the sacrum 11. In other embodiments, the bottom portion 2218 and/or top portion 2219 can have other contours, such as being generally flat. The bottom portion 2218 and top portion 2219 can be compliant and conform to the native anatomy of the surface of the sacrum 11.

FIG. 17D illustrates the tissue retractor 2200 in the collapsed configuration. The inflatable body 2202 can be inflated to retract bowel from the sacrum 11 and to protect the tissue in the presacral space 15 from subsequent instrumentation movement (insertion, actuation, removal) through the channel 2220, as described above in other embodiments. In the illustrated embodiment, the inflatable body 2202 has a generally spherical shape. In some embodiments, the inflatable body 2202 can have a plurality of different types of shapes, such as the shapes discussed above.

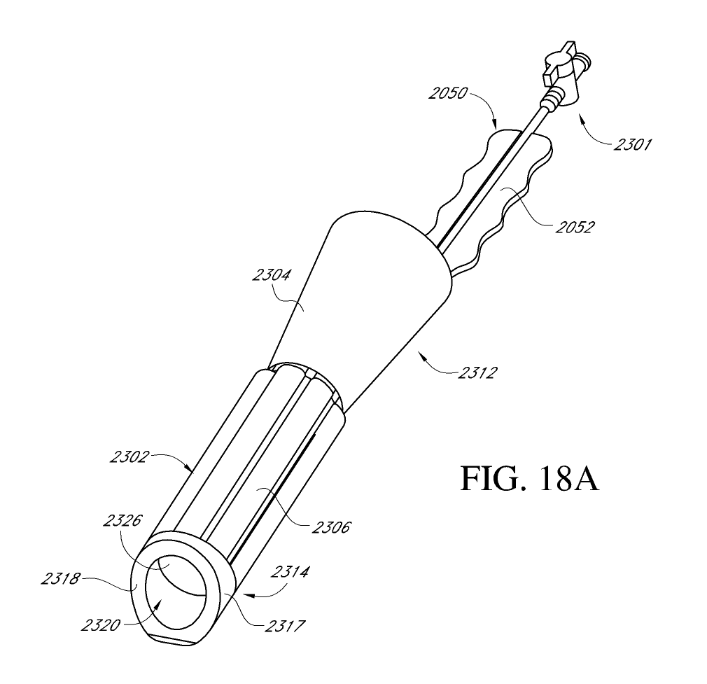

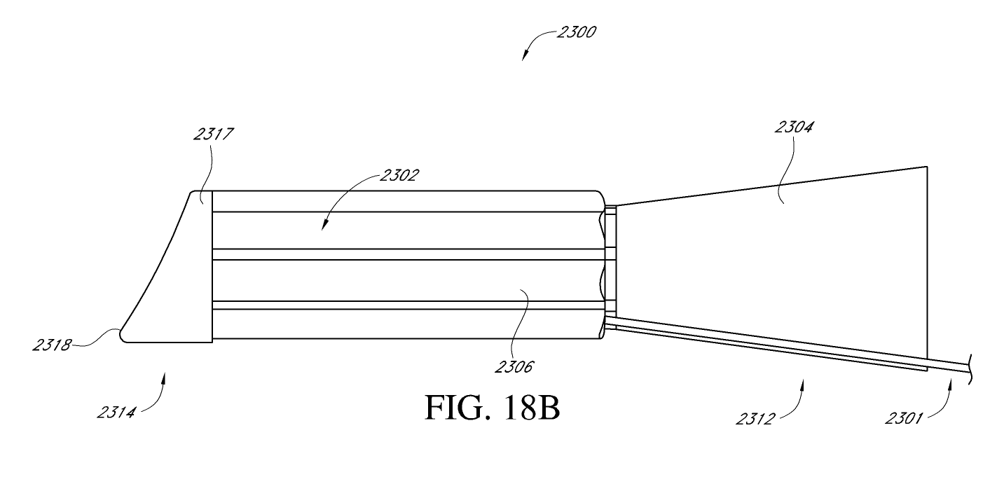

Another embodiment of a tissue retractor 2300 is illustrated in FIGS. 18A-B. The tissue retractor 2300 can have a proximal portion 2312 and a distal portion 2314. The distal portion 2314 can be an inflatable body 2302 that is in fluid communication with an inflation tube 2301, and the proximal portion 2312 can be a funnel 2304, similar to described above. The inflatable body 2302 can have a lumen 2326 that extends from the distal end of the proximal portion 2312 to the distal end of the inflatable body 2302, as illustrated in FIG. 18A. The lumen 2326 can be in fluid communication with the funnel 2304 wherein a channel 2320 can be formed that extends from the proximal end of the funnel 2304 to the distal end of the inflatable body 2302. The inflatable body 2302 can protect the tissue in the presacral space 15 from subsequent instrumentation movement (insertion, actuation, removal) through the channel 2320, as described above in other embodiments. Although the illustrated embodiment is not specifically intended for retracting bowel and/or produces less retraction than the previous embodiments, the body 2302 can achieve retraction of the bowel when inflated. In the illustrated embodiment, the inflatable body 2302 has ribs 2306 that extend in the longitudinal direction. The ribs 2306 can provide rigidity to the inflatable body 2302 when inflated. The inflatable body 2302 can also include a distal tip 2317 that can conform to the surface of the sacrum 11. In some embodiments, the distal tip 2317 can have a tapered surface 2318, as described above, which can abut against the surface of the sacrum 11. The distal tip 2317 can be an inflatable chamber which may or may not be in fluid communication with the rest of the inflatable body 2302. In some embodiments, the distal tip 2317 can be made of a compliant material that can conform to the shape of the sacrum 11.

In various embodiments, the general shape of the tissue retractors and the openings extending there-through can be any shape, such as a rectangular, triangular, or oval. Preferably, the tissue retractor can have a shape that effectively retracts the bowel from the sacrum 11 when inflated and can be easily inserted into the presacral space 15 in the uninflated configuration. In some embodiments, the width of the tissue retractor can be wider at the distal portion compared to the proximal portion to enable retraction of more of the bowel adjacent the target site 192.

An advantage of the embodiment of FIG. 18A and many of the embodiments described herein is the distal tip of the tissue retractor 230 is able to conform to the face of the sacrum. For example, as the device is pushed against the sacrum by a surgeon the distal face of the tissue retractor can conform as the compliant material and inflation media adapt to the shape of the sacrum. This arrangement advantageously reduces gaps between the device and the sacrum and to reduce the pinching and/or intrusion of soft tissue.

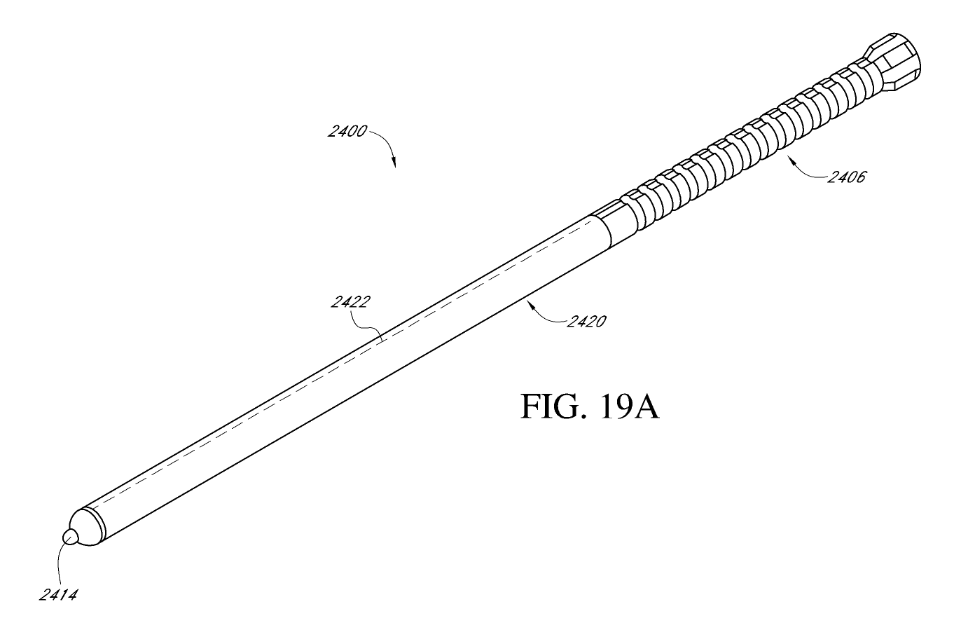

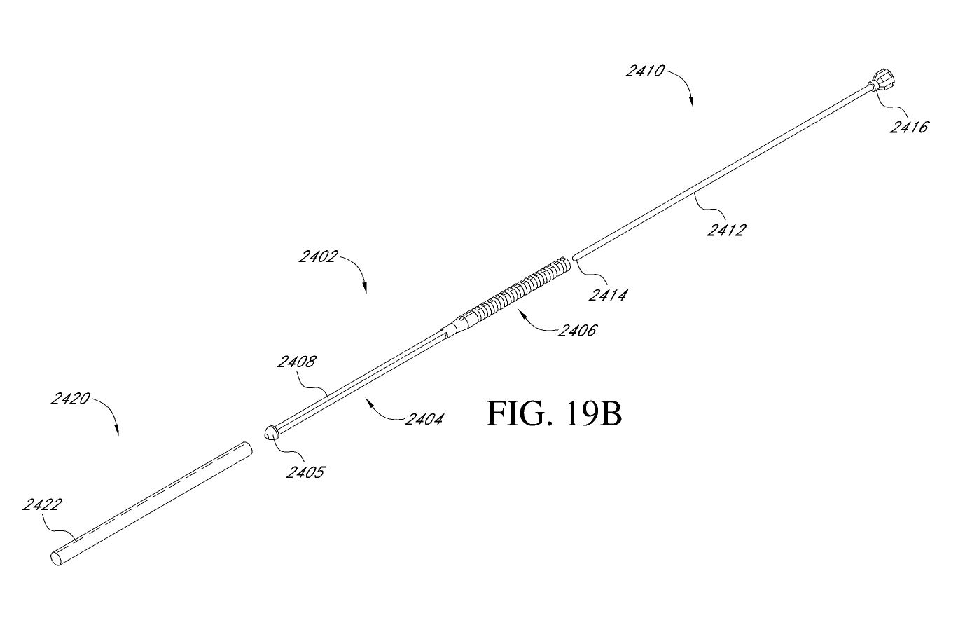

FIGS. 19A-B illustrate an embodiment of an insertion tool 2400 that can be used to position a tissue retractor according to one or more of the embodiments described above into the presacral space 15. The insertion tool 2400 can have an elongate insertion housing 2402 that can be used to deliver the tissue retractor into the presacral space 15. The insertion housing 2402 can have a distal portion 2404 that is configured to hold the tissue retractor for delivery. For example, as illustrated in FIG. 19B, the distal portion 2404 can have one or more grooves 2408 cut into the side of the insertion housing 2402. The groove(s) can provide available area to fit material of the tissue retractor. In some embodiments, the distal portion 2404 can have other types of cavities for accepting the tissue retractor, such as for example spherical cutouts. The wings of the tissue retractor at a lateral side(s) may be wrapped in individual spirals until the lateral sides meet at a lateral side(s) of the insertion tool 2400. In some embodiments, the distal portion 2404 can have a reduced diameter such that when the tissue retractor is wrapped around the distal portion 2404, the overall diameter can be generally the same as the rest of the insertion tool 2400. The distal portion 2404 can have any other features to functionally contain the tissue retractor. The tissue retractor is preferably wrapped around the insertion housing 2402 in a manner such that the tissue retractor can unwrap fully when it is inflated. The tissue retractor can be secured to the insertion housing 2402 toward the distal end 2405 to help prevent the tissue retractor from sliding proximally relative to the insertion housing 2402 during inflation. In some embodiments, the distal end 2405 of the insertion housing 2402 can be curved or rounded to assist insertion through the tissue. The rounded end also helps to avoid injury to the bowel around the presacral space 15.

The proximal portion 2406 of the insertion housing 2402 can be configured for gripping. In some embodiments, the proximal portion 2406 can have a textured surface. For example, as illustrated in FIGS. 19A-B, the proximal portion 2406 can have a plurality of circumferential ridges to help maintain grip as the insertion tool 2400 is moved in the distal-proximal direction. In other embodiments, the proximal portion 2406 can have a plurality of different types of features to enhance gripping of the insertion housing 2402, such as for example rubber coatings or lateral extensions.

The insertion tool 2400 can include a stylet 2410 that is inserted through a channel that extends longitudinally through the insertion housing 2402. The stylet 2410 can be an elongate rod 2412 with an atraumatic or blunt tip 2414 at the distal end. The length of the elongate rod 2412 is sufficiently long such that the tip 2414 extends distally beyond the distal end 2405 of the insertion housing 2402 when the stylet 2410 is coupled with the insertion housing 2402. The blunt tip 2414 can help to dissect through the native anatomy and/or anchor the insertion tool 2400 to the sacrum 11, as described further below. The proximal end of the stylet 2410 can be configured to couple with the proximal end of the insertion housing 2402. For example, the stylet 2410 can have a connector 2416, such as threads, that couple with a complementary connector on the insertion housing 2402. In the illustrated embodiment, the proximal end of the stylet 2410 has gripping features to help in rotating the stylet 2410. In some embodiments, other functional couplers can be used between the stylet 2410 and the insertion housing 2402, such as interference fits.

Once the tissue retractor is placed on the distal portion 2404 of the insertion housing, a sheath 2420 can be placed over the tissue retractor (which can be wrapped uniformly around the distal portion 2404) to contain the tissue retractor and provide a smooth surface during positioning of the insertion tool 2400 in the presacral space 15. For example, the sheath 2420 can hold down the tissue retractor to give the inserter a uniform diameter. In some embodiments, the sheath 2420 can have a perforation line 2422 that creates a weak point in the sheath 2420 so that the sheath 2420 can break apart to release the tissue retractor when the tissue retractor is inflated. As illustrated in FIGS. 19A-B, the perforation line 2422 can extend longitudinally along the sheath 2420. In other embodiments, the perforation line 2422 can extend transversely to the sheath 2420 or in various directions. Preferably, the sheath 2420 is secured to the insertion housing or tissue retractor so that the sheath can be extracted from the presacral space 15 when the procedure is complete.

The introducer assembly 134, the stylet 119 and/or the guide pin (described above) can be inserted before or after the insertion of a tissue retractor according one or more of the embodiments described herein. For example, in one arrangement the introducer assembly 134 and the stylet 119 can be advanced to the face of the sacrum to establish a trajectory. The stylet 119 can be removed and a sharp guide pin can then be introduced and secured into the bone through the introducer assembly 134. The retraction device can then be inserted over the guide pin and/or inserted over the guide pin using one or more of the insertion tools described above. The rectraction device can then be expanded to create the working channel with the guide pin running through over the working channel or surface of the retraction device.

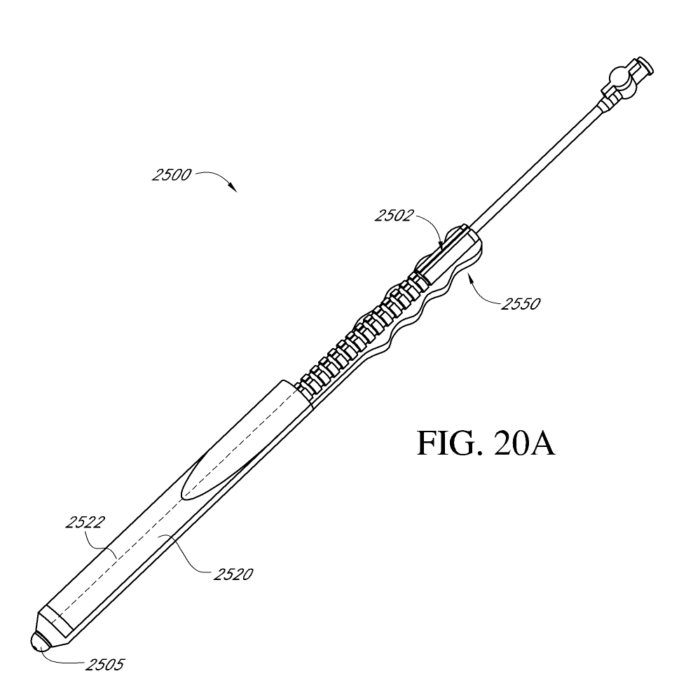

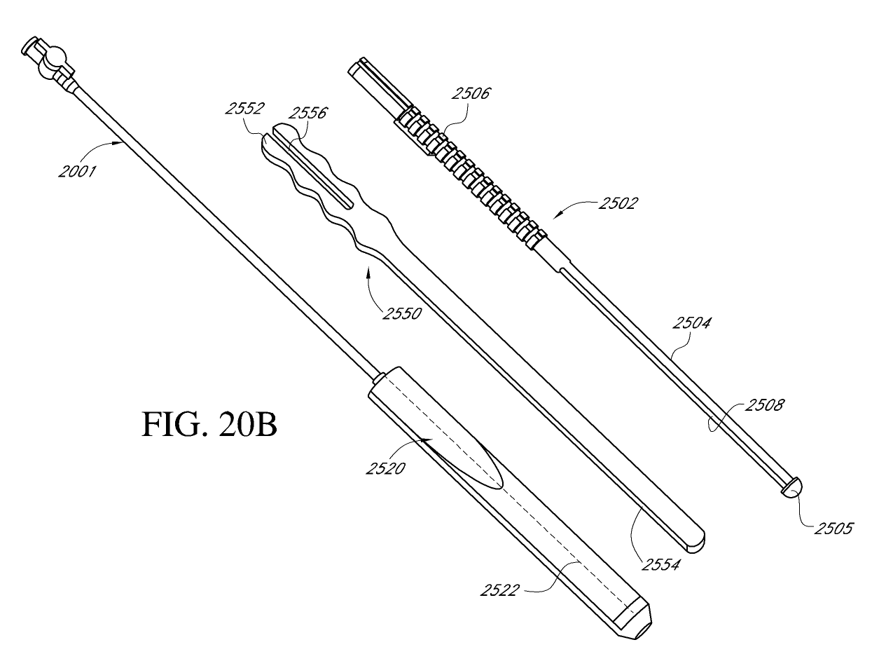

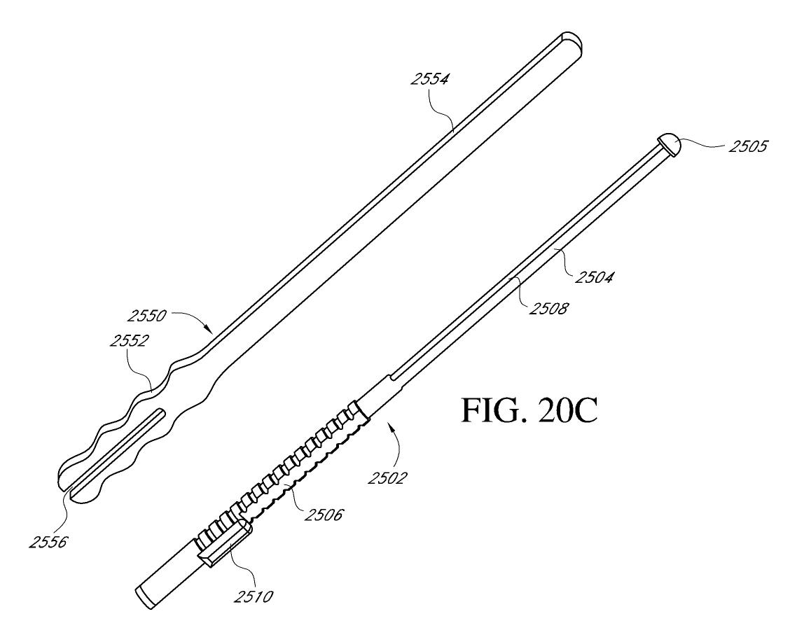

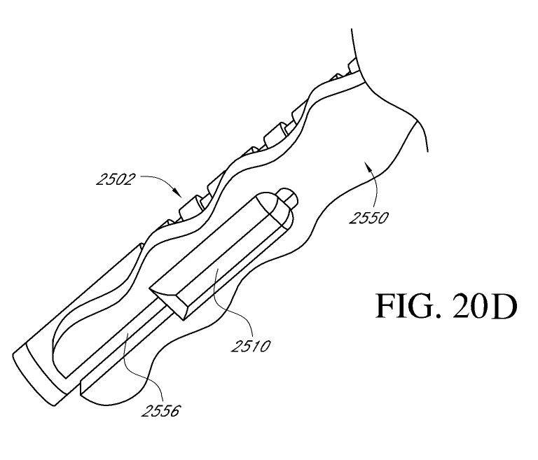

FIGS. 20A-D illustrate another embodiment of an insertion tool 2500 that can be used to position the tissue retractor in the presacral space 15. As illustrated in the assembled view of FIG. 20A, the insertion tool 2500 can include an insertion housing 2502, adjustment tool 2550 and a sheath 2520.

The insertion housing 2502 can be an elongate member that is used to deliver the tissue retractor into the presacral space 15. Similar to other embodiments described above, the insertion housing 2502 can have a distal portion 2504 that is configured to accept the tissue retractor for delivery. In some embodiments, the distal portion 2504 can be configured to hold the tissue retractor. For example, as illustrated in FIG. 20B, the distal portion 2504 can have one or more grooves 2508 cut into the side of the insertion housing 2502. The groove(s) can provide available area to fit tissue retractor material. In some embodiments, the distal portion 2504 can have other types of cavities for accepting the tissue retractor, such as for example spherical cutouts. The lateral sides of the tissue retractor can be wrapped or rolled into compact individual rolls that can be adjacent to the distal portion 2504 of the insertion tool 2500. In some embodiments, the distal portion 2504 can have a reduced diameter such that when the tissue retractor is placed against the distal portion 2504, the overall diameter can be generally the same as the rest of the insertion tool 2500. In other embodiments, the overall diameter of the tissue retractor and the distal portion 2504 can be larger or smaller than the rest of the insertion tool 2500. A sheath 2520 can be wrapped around the tissue retractor to contain the tissue retractor in a compact configuration, as explained further below. Furthermore, the distal portion 2504 can have any other features to functionally contain the tissue retractor. The tissue retractor is preferably wrapped around the insertion housing 2502 in a manner such that the tissue retractor can unwrap fully when it is inflated. The sheath 2520 can be secured to the insertion housing 2502 toward the distal end 2505 to help prevent the tissue retractor from sliding proximally relative to the insertion housing 2502 during inflation. In some embodiments, the distal end 2505 of the insertion housing 2502 can be curved or rounded to assist insertion through the tissue. The rounded end also helps to avoid injury to the bowel around the presacral space 15.

The proximal portion 2506 of the insertion housing 2502 can be configured for gripping. In some embodiments, the proximal portion 2506 can have a textured surface. For example, as illustrated in FIGS. 20A-D, the proximal portion 2506 can have a plurality of circumferential ridges to help maintain grip as the insertion tool 2500 is moved in the distal-proximal direction. In other embodiments, the proximal portion 2506 can have a plurality of different types of features to enhance gripping of the insertion housing 2502, such as for example rubber coatings or lateral extensions.

Once the tissue retractor is placed on the distal portion 2504 of the insertion housing, a sheath 2520 can be placed over the tissue retractor to contain the tissue retractor and provide a smooth surface during positioning of the insertion tool 2500 in the presacral space 15. In some embodiments, the sheath 2520 can have a perforation line 2522 that creates a weak point in the sheath 2520 so that the sheath 2520 can break apart to release the tissue retractor when the tissue retractor is inflated. As illustrated in FIGS. 20A-B, the perforation line 2522 can extend longitudinally along the sheath 2520. In other embodiments, the perforation line 2522 can extend transversely to the sheath 2520 or in various directions. Preferably, the sheath 2520 is secured to the insertion housing or tissue retractor so that the sheath can be extracted from the presacral space 15 when the procedure is complete.

In some embodiments, the insertion tool 2500 can include an adjustment tool 2550 that can be coupled to the tissue retractor to help manipulate and position the tissue retractor within the presacral space 15, as described further below. In some embodiments, the adjustment tool 2550 can have a handle portion 2552 that is configured for gripping. For example, in the illustrated embodiment, the handle portion 2550 includes wavy edges to provide finger grips. A plurality of different types of gripping features can be used. The distal portion 2554 of the adjustment tool 2550 can be thin and elongate. In some embodiments, the insertion tool 2500 with the adjustment tool 2050 can be inserted into the presacral space 15 to position the tissue retractor near the target site 192. The tissue retractor can be inflated and then in some embodiments, the insertion housing 2502 can be removed once the trajectory is established and the guide pin is inserted, leaving the adjustment tool 2550 coupled to the tissue retractor. The distal portion 2554 of the adjustment tool 2550 can fit in a pocket that extends through the tissue retractor under the channel. The adjustment tool 2550 can preferably remain coupled to the tissue retractor for further adjustments during the surgical procedure and to aid in removal of the tissue retractor after the surgical procedure.

As illustrated in FIGS. 20C-D, the insertion housing 2502 and adjustment tool 2550 can have complementary coupling features to hold the two components together during the implant procedure. In the illustrated embodiment, the adjustment tool 2550 has a slot 2556 that extends longitudinally along the handle portion 2552. The insertion housing 2502 has a protrusion on the proximal portion 2506 that is configured to slide in the slot 2556 to couple the insertion housing 2502 to the adjustment tool 2550. The insertion housing 2502 can be removed from the insertion tool 2500 during the implant procedure by sliding the insertion housing 2502 in the proximal direction. In other embodiments, any functional coupler can be used, such as for example hooks, threads, magnets, etc.

In some embodiments, the insertion tool 2500 can include a stylet (not pictured) that is inserted through a channel that extends longitudinally through the insertion housing 2502, as described above in other embodiments. The stylet can be an elongate rod with a blunt or an atraumatic tip at the distal end. The length of the elongate rod is sufficiently long such that the blunt tip extends distally beyond the distal end 2505 of the insertion housing 2502 when the stylet is coupled with the insertion housing 2502. The proximal end of the stylet can be configured to couple with the proximal end of the insertion housing 2502. For example, the stylet can have a connector, such as threads, that couple with a complementary connector on the insertion housing 2502. In some embodiments, other functional couplers can be used between the stylet 2510 and the insertion housing 2502, such as interference fits.

In some embodiments, the insertion housing is cannulated so that the stylet and the insertion tool can be inserted over a guide pin or guidewire already positioned in the patient (e.g., using the techniques described above). In some embodiments, the guide pin can be inserted through the cannulated insertion housing before or after the tissue retraction device is inflated. In other embodiments, the insertion tool is advanced to the target site after (or in place of) a finger dilation and before the use of a guide pin or guide pin assembly. In such embodiments, the guide pin and/or guide assembly can be inserted through or over the tissue retractor.

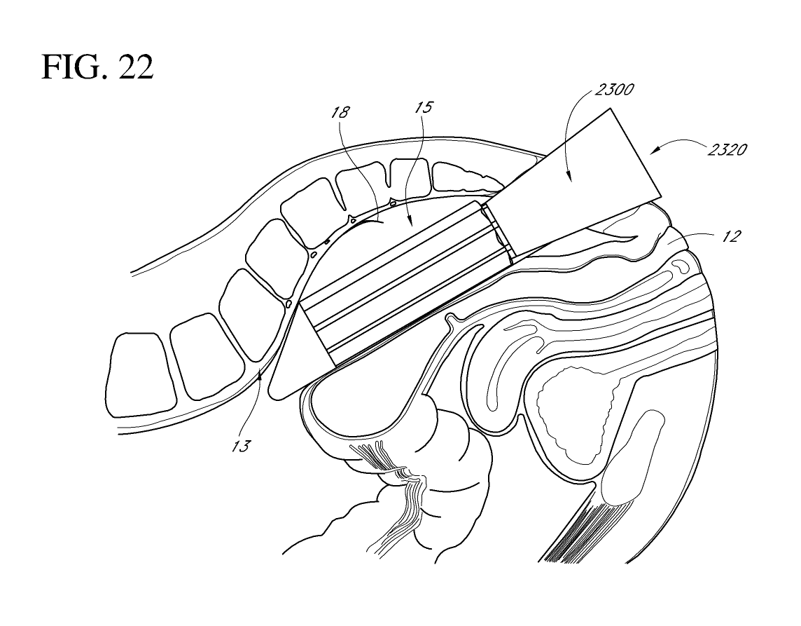

As described previously, a goal of some embodiments of the tissue retractor is to keep the bowel away from the anterior surface 17 and to protect and/or prevent instrumentation that is inserted during the surgical procedure from touching the bowel. In other words, the tissue retractor retracts the bowel away from the anterior surface 17 and forms a working channel for instrumentation to pass without obstruction from the bowel. FIG. 21 illustrates an embodiment of a tissue retractor 2000 positioned in the presacral space 15 with an tubular retractor 704 disposed in the working channel 2022. FIG. 22 illustrates another embodiment of a tissue retractor 2300 positioned in the presacral space 15 and forming a working channel 2320, which can be used without an tubular retractor.

A method of using the tissue retractors will be described with reference to an embodiment of the tissue retractor 2000. However, it should be understood that other embodiments of tissue retractors can be used and the described method is not limited to the specific embodiment described.

In some embodiments, the bowel can be partially dissected from the sacrum 11 as described above (e.g., using finger dissection) and then the tissue retractor 2000 can be introduced into the presacral space 15. In some embodiments, the tissue retractor 2000 can be coupled to a guide pin 100 and the retractor 2000 can be inserted into the presacral space 15 with the guide pin 100. In the uninflated configuration, the tissue retractor 2000 can be yielding and can be inserted easily through the skin incision 40. In preferred embodiments, the tissue retractor 2000 can be compacted by folding or rolling the sides of the tissue retractor 2000 along the proximal distal length, as described above. The folded/rolled configuration allows for easier insertion through the incision 40 and into the presacral space 15. In an embodiment, the sheath and/or tissue retractor 2000 is coated with a surfactant to assist in the insertion and placement of the tissue retractor 2000. For example, in one embodiment, a biocompatible medical coating or surfactant can be applied to the sheath, inserter, and/or tissue retractor 2000 to enhance hydrophilicity and reduce friction between the tissue retractor 2000 and the native anatomy.

In preferred embodiments, the tissue retractor 2000 is positioned to extend from at least the target site 192 to the incision 40, as illustrated in FIG. 21. In one embodiment, with the device inflated, an open channel extends from the target site to the incision such that the surgeon can visualize the target site.

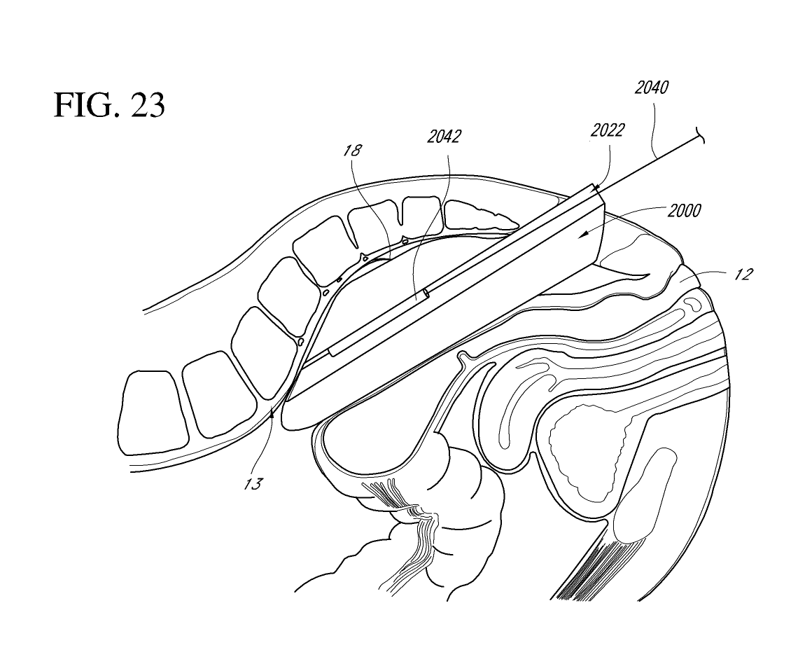

In another embodiment, the tissue retractor 2000 can be inserted into the presacral space 15 over a guide wire 2040 that is anchored to the target site 192, as illustrated in FIG. 23. In some embodiments, the tissue retractor 2000 can have a bushing 2042 or tube to slide over the guide wire 2040. The tissue retractor 2000 can enter through the incision 40 over the guide wire 2040 until it reaches the desired position in the presacral space 15. As described above, in some embodiments, the tissue retractor 2000 can include an inflation tube that is in fluid communication with the body 2002 and can preferably extend outside of the patient for connecting to an inflation media source. Inflation media can be inserted into the inflatable body 2002 to retract the bowel from the sacrum 11. The inflatable body 2002 also forms a working pathway for introduction of exchange an cannula and other instrumentation. This method advantageously allows rapid, safe and efficient achievement of sufficiently large dilation. For example, instead of sequential dilators that may cause trauma with each dilator that is inserted, the tissue retractor 2000 quickly dilates the bowel and forms a working pathway in minimal steps.

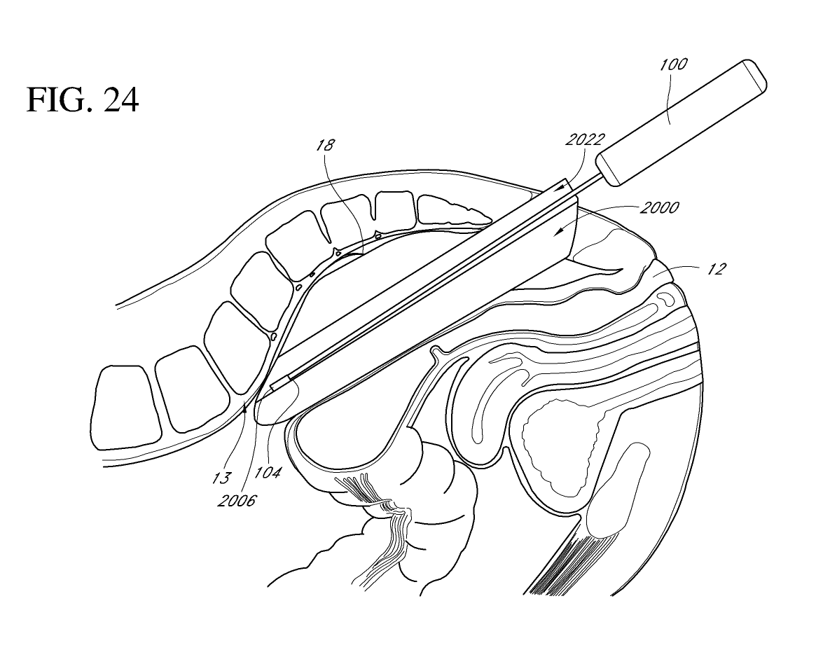

In another embodiment, the tissue retractor 2000 can be coupled to a guide pin 100 for insertion into the presacral space 15, as illustrated in FIG. 24. In the illustrated embodiment, the guide pin 100 has a handle at the proximal end and the distal end 104 is coupled to the tissue retractor 2000. In some embodiments, the tissue retractor 1800 can comprise an interface 2006 within which the distal end 104 of the guide pin 100 can be inserted. In various embodiments, the interface 2006 can be a pocket, a link, a releasable lock, a permanent lock, or other connection. In some embodiments, the interface 2006 can be disposed below the channel 2020. This advantageously can allow the tissue retractor 2000 to be wrapped around the guide pin 100, which minimizes the cross-sectional area of the devices and facilitates controlled deployment. The guide pin 100 with tissue retractor 2000 can be inserted into presacral space to access the target site 192, as described above. In some embodiments, the guide pin 100 can have a sharp tip to puncture the Waldeyer's Fascia 18. Once the target site 192 is reached, the guide pin 100 can be anchored to the sacrum 11 and/or abutted against the sacrum without being anchored. Then, the tissue retractor 2000 can be inflated to retract the bowel from the sacrum 11 and create a working pathway. The guide pin 100 can remain anchored to the tissue retractor 2000 and can be used for manipulation of the tissue retractor 2000 during the surgical procedure. After the surgical methods are performed through the presacral space 15 and the instrumentation are removed, the tissue retractor 2000 can be removed, as described above for the tissue retractor 1800.

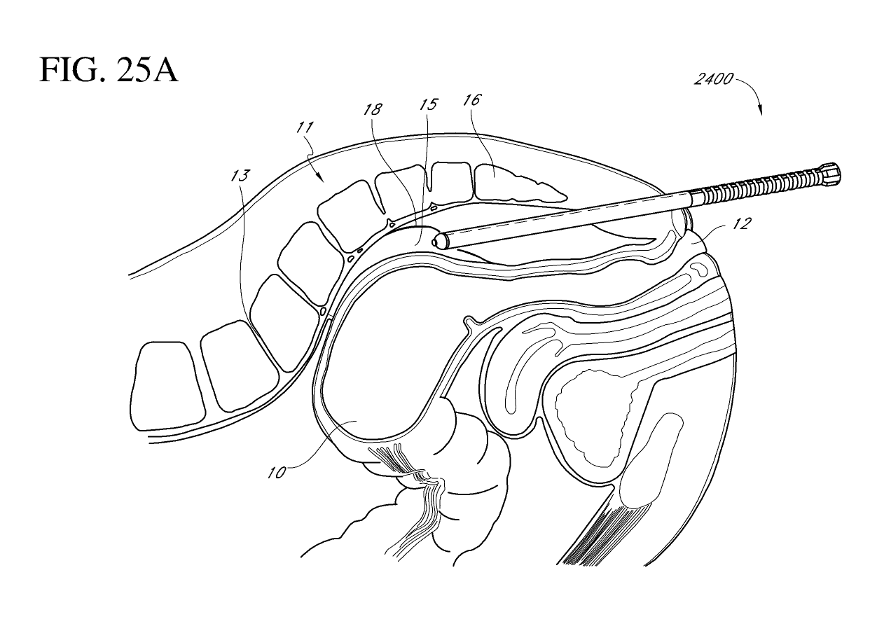

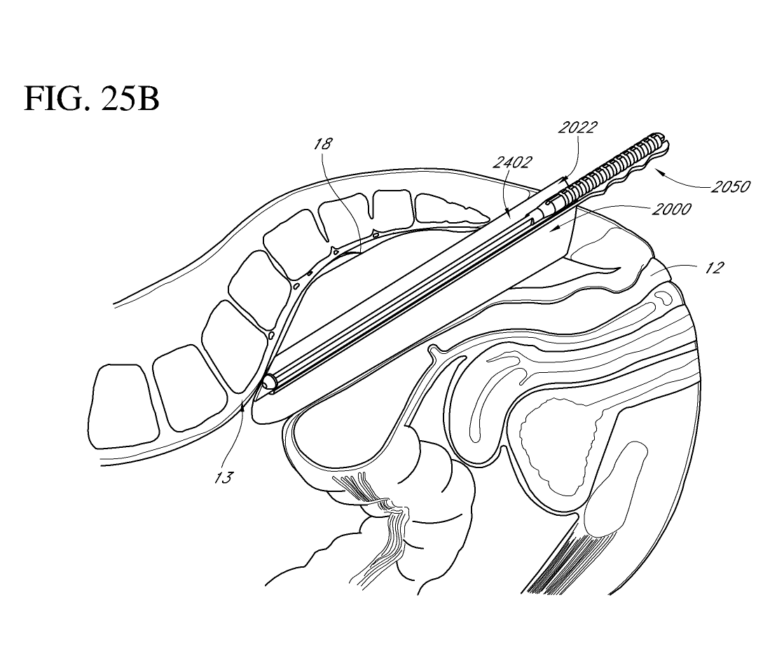

In another embodiment, the insertion tool 2400 can be used to deliver the tissue retractor 2000 into the presacral space 15, as illustrated in FIGS. 25A-B. In the illustrated embodiment, the insertion tool 2400 is inserted into presacral space 15. The pointed tip 2414 of the stylet 2410 can puncture the Waldeyer's Fascia 18 to reach the target site 192, as illustrated in FIG. 25A. In some embodiments, the stylet 2410 can be removed from the insertion housing 2402 after the Waldeyer's Fascia 18 is punctured. Once the target site 192 is reached, the insertion tool 2400 can be anchored to the sacrum 11 and/or abutted against the sacrum without being anchored. Then, the tissue retractor 2000 can be inflated to break open the sheath 2420 and retract the bowel from the sacrum 11 to create a working pathway, as illustrated in FIG. 25B. The insertion tool 2400 can remain anchored to the tissue retractor 2000 and can be used for manipulation of the tissue retractor 2000 during the surgical procedure. After the surgical methods are performed through the presacral space 15 and the instrumentation are removed, the tissue retractor 2000 and insertion tool 2400 can be removed.

In some embodiments, a plurality of inserters may be used. For example, a separate adjustment tool 2050 can be coupled to the tissue retractor 2000 to help manipulate and position the tissue retractor 2000 within the presacral space 15, as illustrated for example in FIGS. 17C-D and 18A. Preferably, the adjustment tool 2050 is made of a semi-flexible material, such as metals, which are either inherently bendable or configured to be bendable (e.g., dimensionally configured to allow the functionally appropriate amount of pliability). In some embodiments, the adjustment tool 2050 can have a handle portion 2052 that is configured for gripping. For example, in the illustrated embodiment, the handle portion 2050 includes wavy edges to provide finger grips. A plurality of different types of gripping features can be used. The distal portion of the adjustment tool 2050 can be thin and elongate, and made of the semi-flexible material. The semi-flexible material allows the adjustment tool 2050 to bend for improved control. For example, when the adjustment tool 2050 is inserted in the presacral space, the handle portion 2552 can be bent (e.g., downward) out of the way to improve access to the working area and reduce obstructions during delivery of other surgical tools. of the tissue retractor 2000 when inflated, but still allows for control and adjustment of the tissue retractor 2000. In some embodiments, the adjustment tool 2050 with the coupled tissue retractor 2000 can be inserted into the presacral space along with the guide pin 100 to reach the target site 192. In some embodiments, the guide pin 100 can be inserted first and the adjustment tool 2050 with the tissue retractor 2000 can be inserted along the guide pin to reach the target site 192. In some embodiments, the adjustment tool 2050 can be inserted separately after the guide pin introducer tube or blunt stylet are removed. In another embodiment, the adjustment tool 2050 can be cannulated so that the guide pin introducer tube or blunt stylet can be coupled to the adjustment tool 2050 through the cannula. As described above in other embodiments, the adjustment tool 2050 can be coupled to the tissue retractor 2000 below the channel 2020 and the tissue retractor 2000 can be wrapped around the adjustment tool 2050. The tissue retractor 2000 can be inflated to retract the bowel form the sacrum 11. In some embodiments, the guide pin 100 can be removed after inflation, but the adjustment tool 2050 can preferably remain coupled to the tissue retractor 2000 for further adjustments during the surgical procedure and to aid in removal of the tissue retractor 2000 after the surgical procedure.

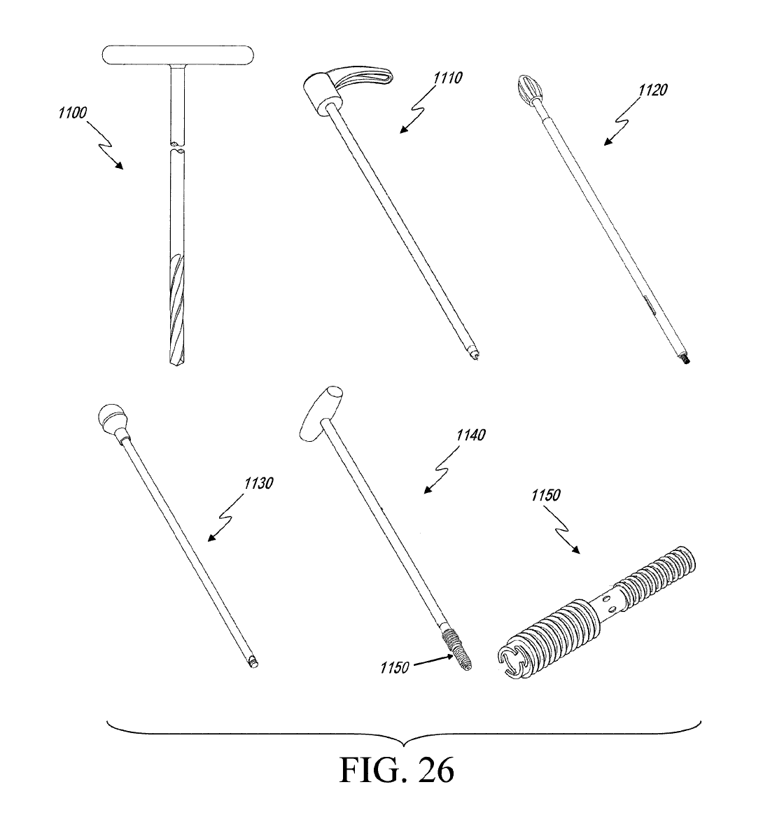

In accordance with aspects of the embodiments described in U.S. patent application Ser. No. 10/972,065, filed on Oct. 22, 2004 (U.S. Patent Publication 2005-0149049) and/or in U.S. patent application Ser. No. 11/501,351, filed on Aug. 9, 2006 (U.S. Patent Publication 2007-0066977), the contents of each of which are incorporated by reference, in their entireties, herein, there is provided various embodiments of surgical tool sets and components configured for insertion and use in a sacral space, presacral space and/or target site at a protected space. In some embodiments, the surgical tools described above and/or in the incorporated references can be used in conjunction with the tissue retractor. For example, FIG. 26 illustrates a twist drill 1100, cutter assembly 1110, tissue extractor 1120, insertion tool assembly 1120, drive tool 1140, and a distraction rod 1150 that can be inserted through a working path defined by the retractors described herein. As noted above, the tubular retractor 704 (see e.g., FIG. 15B) can also be inserted through and/or over the tissue retractors described herein. The tissue retractor can provide a working path in the presacral space 15 for insertion and removal of surgical tools such as the surgical tools shown in FIG. 26 and/or described in (U.S. Patent Publication 2005-0149049) and/or U.S. Patent Publication 2007-0066977 incorporated by reference above. The tissue retractor can also be used to dissect the presacral space 15.

As noted above, in certain embodiments, the tools and devices described above can be arranged into kits (e.g., a common package) or assemblies (e.g., arrangement on a surgical tray) in which the tools and devices are conveniently packaged together and/or used in combination or sequence. For example, in one embodiment, it is advantageous to package a tissue extractor, an insertion tool, one more fixation wires, a tubular retractor (e.g., with a conformable tip) along with an inflation device (e.g., a syringe or inflation bulb). In another embodiment, a package or assembly can include an tissue retractor, an insertion tool and an inflation device (e.g., a syringe or inflation bulb). In another arrangement, a tissue retractor, one or more insertion tools, one or more fixation wires, a tubular retractor (e.g., with a conformable tip) and an inflation device (e.g., a syringe or inflation bulb) can be packaged and/or assembled together. In another arrangement, a tissue retractor can be packaged and/or assembled with more than one insertion tool, and an inflation device. In yet another arrangement, the tissue retractor is packaged and/or assembled together with an introducer and more than one fixation wires, a tubular retractor, an inflation device and a guide pin. In yet another arrangement, the tissue rectractor can be packaged and/or assembled with an insertion tool, an inflation device, and a guide pin. In another embodiment, the tissue retractor can be packaged and/or assembled with more than one insertion tool, more than one fixation wire, more than one tubular retractor, a guide pin and an inflation device. In yet another embodiment, the tissue retractor is packaged and/or assembled together with more than one insertion too, an inflation device and a guide pin. In each of the packages or assemblies described above a guide pin introducer with or without a blunt tip stylet and/or a guide pin.

In many of the embodiments described above, the tissue retraction the device is expanded by inflating the device with an inflation media (e.g., a fluid or gas). However, it should be appreciated that in modified embodiments the retraction device can be expanded without the use of an inflation media. For example, in one embodiment, retraction device can include elongated longitudinal struts that extend through the chambers and/or along the inner or outer surfaces of the retraction device. The struts can be moved away from each other to expand the device from a first small cross-sectional area to a second larger cross-sectional area. In this manner, the device can define a flexible enlarged working cannula through which tools can be inserted as described above.

Thus, embodiments of soft tissue access technology devices and methods have been provided as described above. While the devices, systems, kits and methods have been described in terms of certain specific embodiments, there is no intention to limit the invention to the same. It will be understood that the foregoing is only illustrative of the principles of certain inventions, and that various modifications, omissions, alterations, and combinations can be made by those skilled in the art without departing from the scope and spirit of the invention. Accordingly, it is not intended that the invention be limited, except as by the appended claims.

What is claimed is:

1. A retraction device for retracting bowel from a sacrum, the device comprising a collapsible enclosure that defines a chamber, the collapsible enclosure having a collapsed configuration and an expanded filled configuration when the chamber is filled with a fluid, the expanded filled configuration defining a working channel positioned between the sacrum and the bowel and extending over or through at least a portion of the device.

2. The retraction device of claim 1, wherein the device forms a barrier extending between the sacrum and the bowel to define a working space between the sacrum and the bowel.

3. The retraction device of claim 2, wherein the device in the expanded inflated configuration has a generally flat rectangular shape that has a depth, a width and a length, wherein the depth is less than the width which is less than the length and wherein the length of the collapsible enclosure is oriented along an insertion axis of the device.

4. The retraction device of claim 2, wherein the device in the expanded inflated configuration comprises a distal portion with a generally flat rectangular shape and a proximal portion with a generally flat trapezoidal shape, wherein the collapsible enclosure has a depth, a first width, a second width and a length, wherein the depth is less than the first width which is less than the length, wherein the first width is disposed along the distal portion and is generally constant, wherein the second width is disposed along the proximal portion and is tapered inwardly in the distal to proximal direction, wherein the length of the device is orientated along a longitudinal axis of the elongated inserter.

5. The retraction device of claim 1, in combination with an elongated inserter that has a distal end and a proximal end, the proximal end of the inserter including a handle and the distal end of the inserter coupled to the device.

6. The retraction device of claim 5, further comprising an interface on the device, wherein the inserter can be coupled to the interface

7. The retraction device of claim 6, wherein the collapsible enclosure has a first side and a second side that extend in a longitudinal direction that is generally parallel to a longitudinal axis of the inserter and wherein the inserter is positioned between the first and second sides.

8. The retraction device of claim 5, wherein the first and second sides are folded towards the inserter when the collapsible enclosure is in the collapsed configuration and expand away from the inserter in the inflated configuration.

9. The retraction device of claim 1, further comprising a surgical tool for insertion into the working channel.

10. The retraction device of claim 1, further comprising an elongate tube in fluid communication with the chamber.

11. The retraction device of claim 10, further comprising a valve connected to the elongate tube.

12. The retraction device of claim 1, wherein device forms a plurality of chambers.

13. The retraction device of claim 12, wherein each of the plurality of chambers are connected to elongate tube in fluid communication with the chamber.

14. The retraction device of claim 13, further comprising a valve connected to each elongate tube.

15. The retraction device of claim 1, wherein the chamber is baffled or corrugated.

16. The retraction device of claim 1, wherein the device forms a channel is extending from a proximal end of the device to a distal end of the device in the expanded inflated configuration.

17. The retraction device of claim 16, wherein the channel has a generally U-shaped cross-section.

18. The retraction device of claim 16, wherein the channel is generally tubular in shape.

19. The retraction device of claim 16, further comprising a funnel-shaped proximal portion that is in fluid communication with the channel at a proximal end of the device.

20. The retraction device of claim 16, further comprising inflatable rib structures.

21. The retraction device of claim 16, wherein at least a portion of a distal end of the device forms a beveled surface and the channel forms an opening in the beveled surface.

22. The retraction device of claim 1, in combination with a tubular retractor comprising a malleable distal tip.

23. The retraction device of claim 1, wherein a distal face of the device is conformable to the sacrum.

24. The retraction device of claim 1, wherein a distal face of the device is beveled without respect to a longitudinal axis of the device.

25. A method of providing access to a target site on a sacrum of a patient; the method comprising the steps of:
inserting a retraction device comprising an inflatable structure into a presacral space between a bowel of the patient and the sacrum;
expanding the inflatable structure with an inflation media, such that the inflatable enclosure expands to retract the bowel from the sacrum to create a working space; and
inserting a surgical tool into the working space created between at least portion of the inflatable structure and the sacrum.

26. The method of claim 25, further advancing a guide pin into the presacral space towards the sacrum.

27. The method of claim 26, wherein the guide pin is advanced into the prescral space after the retraction device is inserted into the presacral space.

28. The method of claim 26, wherein the guide pin is advanced into the prescral space before the retraction device is inserted into the presacral space.

29. The method of claim 25, further comprising the step of repositioning the inflatable structure after the inflatable structure is expanded.

30. The method of claim 25 further comprising the steps of:
at least partially emptying the inflatable structure of the inflation media;
repositioning the inflatable structure within the presacral space; and
refilling the inflatable structure with inflation media.

31. The method of claim 25, wherein the step of inserting the inflatable structure into a presacral space comprises positioning the inflatable structure adjacent a sacral promontory.

32. The method of claim 25, wherein the step of inserting a surgical tool into the working space created between the inflatable structure and the sacrum comprises inserting the surgical tool through a channel formed at least partially by the inflatable device.

33. The method of claim 25, further comprising dissecting the presacral space before the step of inserting the retraction device.

34. The method of claim 25, wherein expanding the inflatable structure dissects the presacral space.

35. A method of providing access to a target site on the sacrum of the patient, the method comprising the steps of:
inserting an inserter carrying an inflatable device into a presacral space between a bowel and a sacrum such that a valve and at least a portion of an inflation tube coupled to the inflatable device;
attaching the valve to a source of inflation media; and
filling the inflatable device with the inflation media to expand the collapsible enclosure and retract the bowel from the sacrum.

36. The method of claim 35, further comprising repositioning the inflatable device within the presacral space by manipulating the inserter.

37. The method of claim 35, further comprising the steps of:
at least partially emptying the inflatable device of the inflation media;
repositioning the inflatable device within the presacral space by manipulating the inserter;
refilling the chamber with inflation media; and
removing the inserter from the inflation device.

38. The method of claim 35, wherein the step of inserting the inflatable device into a presacral space comprises positioning the inflatable device adjacent a sacral promontory.

39. The method of claim 35, comprising inserting a surgical tool into the space created between the inflatable device and the sacrum.

40. The method of claim 35, comprising inserting a surgical tool into a channel created extending through at least a portion of the inflatable device

41. A method of providing access to a target site on a sacrum of a patient; the method comprising the steps of:
inserting at least part of a retraction device comprising an inflatable structure that defines a channel into a presacral space between a bowel of the patient and the sacrum; and
inflating the inflatable structure with an inflation media, such that the inflatable enclosure expands to retract the bowel from the sacrum and enlarge the channel defined through the retraction device to define a working space.

42. The method of claim 41, further comprising the step of inserting a surgical tool through the channel to the sacrum.

43. The method of claim 41, wherein the step of inserting at least a part of the retraction device further comprises sliding the retraction device over a guide wire.

44. The method of claim 41, wherein the step of inserting at least a part of the retraction device further comprises using a control tool coupled to the retraction device to adjust the position of the retraction device.

45. A kit for providing retracting bowel from a sacrum, the kit comprising an inflatable retraction device, an inflation device, an insertion tool, a tubular retractor and a fixation wire.
